(19) United States
(12) Patent Application Publication
(10) Pub. No.: US 2011/0086899 A1
(43) Pub. Date: Apr. 14, 2011

(54) PHARMACEUTICAL COMPOSITIONS FOR ORAL ADMINISTRATION

(75) Inventors: Conrad Stewart Winters, Vancouver (CA); Shaun Lee Gammill, Austin, TX (US)

(73) Assignee: XENON PHARMACEUTICALS INC., Burnaby (CA)

(21) Appl. No.: 12/905,048

(22) Filed: Oct. 14, 2010

Related U.S. Application Data
(60) Provisional application No. 61/251,340, filed on Oct. 14, 2009.

(51) Int. Cl. A61K 31/407; A61P 29/00; A61P 31/18; A61P 25/00; A61P 25/06; A61P 19/02; A61P 9/00; A61P 35/00

(52) U.S. Cl. 514/409

(57) ABSTRACT

This invention is directed to pharmaceutical compositions for oral administration to a mammal, wherein the pharmaceutical compositions comprise a spiro-oxindole compound, as a single stereoisomer or as a mixture thereof, or a pharmaceutically acceptable salt thereof. These pharmaceutical compositions are useful for the treatment and/or prevention of sodium channel-mediated diseases or conditions, such as pain.

### CROSS-REFERENCE TO RELATED APPLICATION

This application claims the benefit under 35 U.S.C. §119(e) of U.S. Provisional Patent Application No. 61/251,340, filed Oct. 14, 2009. This application is incorporated herein by reference in its entirety.

### FIELD OF THE INVENTION

The present invention is directed to pharmaceutical compositions for oral administration to a mammal, comprising one or more pharmaceutically acceptable excipients and a therapeutically effective amount of a compound of formula (I-S). In particular, this invention is directed to pharmaceutical compositions for oral administration to a mammal, wherein the pharmaceutical compositions comprise one or more pharmaceutically acceptable excipients and a therapeutically effective amount of a spiro-oxindole compound that is a sodium channel blocker. The pharmaceutical compositions of the invention are therefore useful in treating sodium channel-mediated diseases or conditions, such as pain, including dental pain and primary/inherited erythromelalgia, as well as other diseases and conditions, such as benign prostatic hyperplasia, pruritis, and cancer.

### BACKGROUND OF THE INVENTION

PCT Published Patent Application No. WO 06/110917 is directed to compounds which are disclosed as being useful as sodium channel blockers. These compounds inhibit sodium ion flux through a voltage-dependent sodium channel. As such, the compounds are sodium channel blockers and are therefore useful for treating diseases and conditions in mammals, which are the result of aberrant voltage-dependent sodium channel biological activity or which may be ameliorated by modulation of voltage-dependent sodium channel biological activity. Such diseases and conditions include, but are not limited to, pain such as dental pain and primary/inherited erythromelalgia, central nervous conditions such as epilepsy, anxiety, depression and bipolar disease; cardiovascular conditions such as arrhythmias, atrial fibrillation and ventricular fibrillation; neuromuscular conditions such as restless leg syndrome and muscle paralysis or tetanus; neuroprotection against stroke, neural trauma and multiple sclerosis; and channelopathies such as erythromelalgia and familial rectal pain syndrome.

The compounds disclosed in PCT Published Patent Application No. WO 2006/110917, which is incorporated in full by reference herein, are also useful in treating benign prostatic hyperplasia (BPH), cancer and pruritis (itch).

There exists, therefore, a need to provide suitable pharmaceutical compositions comprising these compounds, particularly for those compounds which demonstrate a low aqueous solubility, for oral administration to mammals in need thereof.

### SUMMARY OF THE INVENTION

The present invention is directed to pharmaceutical compositions comprising one or more pharmaceutically acceptable excipients and a therapeutically effective amount of a spiro-oxindole compound. In particular, the pharmaceutical compositions of the invention are useful in treatment and/or prevention of sodium channel-mediated diseases or conditions and are orally administered to a mammal in need thereof.

Accordingly, in one aspect, the invention is directed to a pharmaceutical composition for oral administration to a mammal, wherein the pharmaceutical composition comprises one or more pharmaceutically acceptable excipients and a therapeutically effective amount of a spiro-oxindole compound having the following formula:

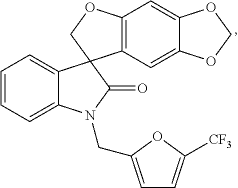

as a racemate, a single enantiomer, or a non-racemic mixture of enantiomers, or a pharmaceutically acceptable salt thereof.

The pharmaceutical compositions of the invention are useful for the treatment of sodium channel-mediated diseases or conditions, including, but not limited to, pain of any nature, including but not limited to, pain associated with HIV, HIV treatment induced neuropathy, trigeminal neuralgia, post-herpetic neuralgia, diabetic neuropathy, peripheral neuropathy, Complex regional pain syndrome, Paroxysmal Extreme Pain Disorder, eudynia, familial erythromelalgia, secondary erythromelalgia, primary/inherited erythromelalgia, familial rectal pain, familial facial pain, dental pain, migraine, headache, familial hemiplegic migraine, sinus headache, tension headache, phantom limb pain, peripheral nerve injury, pain associated with multiple sclerosis (MS); myasthenia syndromes, myotonia, paroxysmal dystonia, periodic paralysis, spasticity, spastic paraplegia, myopathies, paramyotonia congentia, hyperkalemic periodic paralysis, hypokalemic periodic paralysis, malignant hyperthermia, heat sensitivity, irritable bowel syndrome, Crohns disease, motor impairment associated with MS, amyotrophic lateral sclerosis (ALS), pruritis, benign prostatic hyperplasia, arthritis, rheumatoid arthritis, osteoarthritis, cystic fibrosis, pseudoaldosteronism, rhabdomyolysis, bipolar depression, anxiety, schizophrenia, illness due to exposure to insecticides or other sodium channel toxins, cancer, epilepsy, partial and general tonic seizures, restless leg syndrome, fibromyalgia, neuroprotection under ischaemic conditions caused by stroke, glaucoma or neural trauma, arrhythmias, long-QT syndrome, Catecholeminergic polymorphic ventricular tachycardia, tachy-arrhythmias, atrial fibrillation and ventricular fibrillation, wherein the method comprises orally administering to the mammal in need thereof a therapeutically effective amount of a pharmaceutical composition of the invention, as set forth above in the Summary of the Invention.

Accordingly, in another aspect, the invention provides a method for the treatment of pain in a mammal, wherein the method comprises orally administering to the mammal in need thereof a therapeutically effective amount of a pharmaceutical composition of the invention as set forth above.

In another aspect, the present invention provides a method for treating or lessening the severity of a disease, condition, or disorder where activation or hyperactivity of one or more of Naᵥ1.1, Naᵥ1.2, Naᵥ1.3, Naᵥ1.4, Naᵥ1.5, Naᵥ1.6, Naᵥ1.7, Naᵥ1.8, or Naᵥ1.9 is implicated in the disease state, wherein the methods comprise orally administering to the mammal in need thereof a therapeutically effective amount of a pharmaceutical composition of the invention as set forth above.

In another aspect, the invention provides a method of treating a range of sodium channel-mediated diseases or conditions, including, but not limited to, pain of any nature, including but not limited to, pain associated with HIV, HIV treatment induced neuropathy, trigeminal neuralgia, post-herpetic neuralgia, diabetic neuropathy, peripheral neuropathy, Complex regional pain syndrome, Paroxysmal Extreme Pain Disorder, eudynia, familial erythromelalgia, secondary erythromelalgia, primary/inherited erythromelalgia, familial rectal pain, familial facial pain, dental pain, migraine, headache, familial hemiplegic migraine, sinus headache, tension headache, phantom limb pain, peripheral nerve injury, pain associated with multiple sclerosis (MS); myasthenia syndromes, myotonia, paroxysmal dystonia, periodic paralysis, spasticity, spastic paraplegia, myopathies, paramyotonia congentia, hyperkalemic periodic paralysis, hypokalemic periodic paralysis, malignant hyperthermia, heat sensitivity, irritable bowel syndrome, Crohns disease, motor impairment associated with MS, amyotrophic lateral sclerosis (ALS), pruritis, benign prostatic hyperplasia, arthritis, rheumatoid arthritis, osteoarthritis, cystic fibrosis, pseudoaldosteronism, rhabdomyolysis, bipolar depression, anxiety, schizophrenia, illness due to exposure to insecticides or other sodium channel toxins, cancer, epilepsy, partial and general tonic seizures, restless leg syndrome, fibromyalgia, neuroprotection under ischaemic conditions caused by stroke, glaucoma or neural trauma, arrhythmias, long-QT syndrome, Catecholeminergic polymorphic ventricular tachycardia, tachy-arrhythmias, atrial fibrillation and ventricular fibrillation, wherein the method comprises orally administering to the mammal in need thereof a therapeutically effective amount of a pharmaceutical composition of the invention, as set forth above in the Summary of the Invention, wherein the method comprises orally administering to the mammal in need thereof a therapeutically effective amount of a pharmaceutical composition of the invention as set forth above.

In another aspect, the invention provides a method of treating a range of sodium channel-mediated disease or condition in a mammal through inhibition of ion flux through a voltage-dependent sodium channel in the mammal, wherein the method comprises orally administering to the mammal in need thereof a therapeutically effective amount of a pharmaceutical composition of the invention as set forth above.

In another aspect, the invention provides a method of treating or preventing benign prostatic hyperplasia in a mammal, wherein the methods comprise orally administering to the mammal in need thereof a therapeutically effective amount of a pharmaceutical composition of the invention as set forth above.

In another aspect, the invention provides a method of treating or preventing pruritis in a mammal, wherein the method comprises orally administering to the mammal in need thereof a therapeutically effective amount of a pharmaceutical composition of the invention as set forth above.

In another aspect, the invention provides a method of treating or preventing cancer in a mammal, wherein the method comprises orally administering to the mammal in need thereof a therapeutically effective amount of a pharmaceutical composition of the invention as set forth above.

In another aspect, the invention provides for a use of the spiro-oxindole compound for the preparation of a pharmaceutical composition for treating a sodium channel-mediated disease or condition, such as pain, in a mammal, wherein the pharmaceutical composition is prepared for oral administration.

In another aspect, the invention provides a process for the preparation of a pharmaceutical composition of the invention as set forth above.

Specific embodiments of these aspects of the invention are described in more detail below.

### BRIEF DESCRIPTION OF THE DRAWING

The following drawing forms part of the present specification and is included to further demonstrate certain aspects of the present invention. The invention may be better understood by reference to this drawing in combination with the detailed description of specific embodiments presented herein.

FIG. 1 shows the plasma concentration-time profile for COMPOUND B when administered orally to dogs as a single dose of 100 mg or as a single dose of 400 mg (four 100 mg capsules).

### DETAILED DESCRIPTION OF THE INVENTION

### Definitions

Unless defined otherwise in the specification, the following terms and phrases shall have the following meaning:

The term “spiro-oxindole compound” as used herein refers to a compound having the following formula (I):

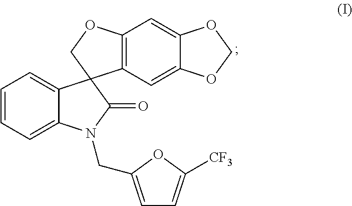

and is intended to include the racemate, both (S) and (R) enantiomers and any non-racemic mixtures of the (S) and (R) enantiomers, and any pharmaceutically acceptable salt thereof. The racemate and any non-racemic mixtures of the (S) and (R) enantiomers of the spiro-oxindole compound is identified herein as COMPOUND A and has the chemical name of 1′-{[5-(trifluoromethyl)-2-furyl]methyl}spiro[furo[2,3-f][1,3]benzodioxole-7,3′-indol]-2′(1′H)-one (as the free base). The preparation of COMPOUND A, or a pharmaceutically acceptable salt thereof, is disclosed in PCT Published Patent Application No. WO 2006/110917, the disclosure of which is incorporated in full by reference herein. The (S)-enantiomer of the spiro-oxindole compound, i.e., the enantiomer having the following formula (I-S):

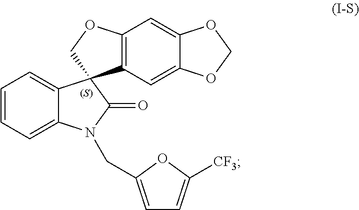

is identified herein as COMPOUND B and has the chemical name of (S)-1′-{[5-(trifluoromethyl)-2-furyl]methyl}spiro[furo[2,3-f][1,3]benzodioxole-7,3′-indol]-2′(1′-H)-one (as the free base). COMPOUND B may be prepared by methods known to one skilled in the art (e.g., by resolution of COMPOUND A by chiral high pressure liquid chromatography) or by the methods described herein. COMPOUND A and COMPOUND B, or a pharmaceutically acceptable salt of either, may also be identified herein as an “active ingredient” of the pharmaceutical compositions of the invention.

“Acceptable Daily Intake” or “ADI” is a measure of the amount of a specific excipient in a pharmaceutical composition that can be ingested (orally) over a lifetime without an appreciable health risk. ADIs are expressed by body mass, usually in milligrams (of the excipient) per kilograms of body mass per day

The term “about” when placed before a numerical value “X” refers in the current application to an interval extending from X minus 10% of X to X plus 10% of X and preferably to an interval extending from X minus 5% of X to X plus 5% of X.

The expression “% w/w” refers to a percentage by weight compared to the total weight of the composition being considered.

“Clathrates” refers to substances which fix gases, liquids or compounds as inclusion complexes so that the complex may be handled in solid form and the included constituent (or “guest” molecule) subsequently releases by the action of a solvent or by melting. The term “clathrate” can be used interchangeably with the phrase “inclusion molecule” or with the phrase “inclusion complex”. Clathrates contemplated for use in the instant invention are prepared from cyclodextrins. Cyclodextrins are widely known as having the ability to form clathrates (i.e., inclusion compounds) with a variety of molecules. See, for example, Inclusion Compounds, edited by J. L. Atwood, J. E. D. Davies, and D. D. MacNicol, London, Orlando, Academic Press, 1984; Goldberg, I., “The Significance of Molecular Type, Shape and Complementarity in Clathrate Inclusion”, Topics in Current Chemistry (1988), Vol. 149, pp. 2-44; Weber, E. et al., “Functional Group Assisted Clathrate Formation—Scissor-Like and Roof-Shaped Host Molecules”, Topics in Current Chemistry (1988), Vol. 149, pp. 45-135; and MacNicol, D. D. et al., “Clathrates and Molecular Inclusion Phenomena”, Chemical Society Reviews (1978), Vol. 7, No. 1, pp. 65-87. Conversion into cyclodextrin clathrates is known to increase the stability and solubility of certain compounds, thereby facilitating their use as pharmaceutical agents. See, for example, Saenger, W., “Cyclodextrin Inclusion Compounds in Research and Industry”, Angew. Chem. Int. Ed. Engl. (1980), Vol. 19, pp. 344-362; U.S. Pat. No. 4,886,788 (Schering A G); U.S. Pat. No. 6,355,627 (Takasago); U.S. Pat. No. 6,288,119 (Ono Pharmaceuticals); U.S. Pat. No. 6,14,969 (Ono Pharmaceuticals); U.S. Pat. No. 6,235,780 (Ono Pharmaceuticals); U.S. Pat. No. 6,262,293 (Ono Pharmaceuticals); U.S. Pat. No. 6,225,347 (Ono Pharmaceuticals); and U.S. Pat. No. 4,935,446 (Ono Pharmaceuticals).

A “mammal” refers to humans or any animals including, but not limited to, mammals of the Orders Primate (including humans, apes and monkeys), Arteriodactyla (including horses, goats, cows, sheep, pigs), Rodenta (including mice, rats, rabbits, and hamsters), and Carnivora (including cats, and dogs). Among birds, the mammals include, but are not limited to, turkeys, chickens and other members of the same order. In specific embodiments, the recipients are humans as the intended use of the invention formulation is human pharmaceutical applications. In addition, the invention formulation can also be suitable for veterinary applications without further manipulations that changes the excipients or excipient ratios that are present.

“Pharmaceutically acceptable excipient” includes without limitation any solvent, adjuvant, bioavailability enhancer, carrier, glidant, sweetening agent, diluent, preservative, dye/colorant, flavor enhancer, solubilizer (including surfactants), wetting agent, dispersing agent, suspending agent, stabilizer, isotonic agent, buffer or emulsifier which has been approved by the United States Food and Drug Administration, Health Canada or the European Medicines Agency, as being acceptable for use in humans or domestic animals.

“Pharmaceutically acceptable salt” includes both acid and base addition salts.

“Pharmaceutically acceptable acid addition salt” refers to those salts which retain the biological effectiveness and properties of the free bases, which are not biologically or otherwise undesirable, and which are formed with inorganic acids such as, but are not limited to, hydrochloric acid, hydrobromic acid, sulfuric acid, nitric acid, phosphoric acid and the like, and organic acids such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, undecylenic acid, and the like.

“Pharmaceutically acceptable base addition salt” refers to those salts which retain the biological effectiveness and properties of the free acids, which are not biologically or otherwise undesirable. These salts are prepared from addition of an inorganic base or an organic base to the free acid. Salts derived from inorganic bases include, but are not limited to, the sodium, potassium, lithium, ammonium, calcium, magnesium, iron, zinc, copper, manganese, aluminum salts and the like. Preferred inorganic salts are the ammonium, sodium, potassium, calcium, and magnesium salts. Salts derived from organic bases include, but are not limited to, salts of primary, secondary, and tertiary amines, substituted amines including naturally occurring substituted amines, cyclic amines and basic ion exchange resins, such as ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, N-ethylpiperidine, polyamine resins and the like. Particularly preferred organic bases are isopropylamine, diethylamine, ethanolamine, trimethylamine, dicyclohexylamine, choline and caffeine.

A “pharmaceutical composition” refers to a formulation of an active ingredient and a medium generally accepted in the art for the delivery of the active ingredient to mammals, e.g., humans or animals. Such a medium includes all pharmaceutically acceptable excipients. For purposes of this disclosure, the phrase “pharmaceutical composition” is interchangeable with the phrase “pharmaceutical formulation”.

“Therapeutically effective amount” refers to that amount of an active ingredient or that amount of a pharmaceutical composition of the invention which, when administered to a mammal, preferably a human, is sufficient to effect treatment, as defined below, of the indicated disease or condition in the mammal. The amount of the active ingredient or the pharmaceutical composition which constitutes a “therapeutically effective amount” will vary depending on the active ingredient, the pharmaceutical composition, the disease or condition and its severity, other conditions affecting the health of the mammal to be treated, the manner of administration, and the age of the mammal to be treated, but can be determined routinely by one of ordinary skill in the art having regard to his own knowledge and to this disclosure.

“Treating” or “treatment” as used herein covers the treatment of the disease or condition of interest in a mammal, preferably a human, having the disease or condition of interest, and includes:

(i) preventing the disease or condition from occurring in a mammal, in particular, when such mammal is predisposed to the condition but has not yet been diagnosed as having it;

(ii) inhibiting the disease or condition, i.e., arresting its development;

(iii) relieving the disease or condition, i.e., causing regression of the disease or condition; or

(iv) relieving the symptoms resulting from the disease or condition.

As used herein, the terms “disease” and “condition” may be used interchangeably or may be different in that the particular malady or condition may not have a known causative agent (so that etiology has not yet been worked out) and it is therefore not yet recognized as a disease but only as an undesirable condition or syndrome, wherein a more or less specific set of symptoms have been identified by clinicians.

### Embodiments of the Invention

Of the various aspects of the invention set forth above in the Summary of the Invention, certain embodiments are preferred.

One embodiment of the pharmaceutical compositions of the invention, as set forth above in the Summary of the Invention, is a pharmaceutical composition comprising two or more pharmaceutically acceptable excipients.

Another embodiment is a pharmaceutical composition wherein the one or more pharmaceutically acceptable excipients are selected from the group consisting of Miglyol® 840, Labrafac®, Captex® 200P, Myvacet® 9-45K, PEG 400, Capmul® PG8, TPGS, Neobee® M-5, Transcutol®, Capryol® 90, Solutol® HS 15, Corn Oil Labrasol®, Capryol® 90, Gelucire® 44/14, a cyclodextrin, PEG 400, PEG 6000, ethanol, water, propylene glycol, Cremophor ELP®, Imwitor® 742, Vitamin E and Polyvinylpyrrolidone (PVP).

Another embodiment is a pharmaceutical composition wherein the one or more pharmaceutically acceptable excipients are selected from the group consisting of Labrasol®, Gelucire® 44/14 and propylene glycol.

Another embodiment is a pharmaceutical composition wherein the one or more pharmaceutically acceptable excipients are selected from the group consisting of Labrasol®, Cremophor® ELP, Imwitor® 742, Vitamin E and PVP.

Another embodiment is a pharmaceutical composition wherein each of the one or more pharmaceutically acceptable excipients are present in a concentration of from about 0.1% w/w to about 99% w/w.

Another embodiment is a pharmaceutical composition wherein the one or more pharmaceutically acceptable exicipients are selected from the group consisting of Labrasol®, Gelucire® 44/14 and propylene glycol and wherein Labrasol® is present in a concentration of from about 30% to about 70% w/w, Gelucire® 44/14 is present in a concentration of from about 20% to about 50% w/w and propylene glycol is present in a concentration of from about 0.5% to about 20% w/w.

Another embodiment is a pharmaceutical composition wherein the one or more pharmaceutically acceptable exicipients are selected from the group consisting of Labrasol®, Cremophor ELP®, Imwitor® 742, Vitamin E and PVP and wherein Labrasol® is present in a concentration of from about 30% to about 70% w/w, Cremophor ELP® is present in a concentration of from about 20% to about 50% w/w, Imwitor® 742 is present in a concentration of from about 0.5% to about 10% w/w, Vitamin E is present in a concentration of from about 0.1% to about 5% w/w and PVP is present in a concentration of from about 0.5% to about 10% w/w.

Another embodiment is a pharmaceutical composition wherein the one or more pharmaceutically acceptable exicipients are selected from the group consisting of Labrasol®, Gelucire® 44/14 and propylene glycol and wherein Labrasol® is present in a concentration of from about 35% to about 65% w/w, Gelucire® 44/14 is present in a concentration of from about 25% to about 45% w/w and propylene glycol is present in a concentration of from about 1.0% to about 10% w/w.

Another embodiment is a pharmaceutical composition wherein the spiro-oxoindole compound is present in a concentration of from about 0.1% w/w to about 25% w/w.

Another embodiment is a pharmaceutical composition in a capsule form containing the spiro-oxindole compound in a unit dosage amount of between about 5 mg to about 100 mg.

Another embodiment is a pharmaceutical composition wherein the pharmaceutical composition is in liquid form.

Another embodiment is a pharmaceutical composition for oral administration to a mammal comprising one or more pharmaceutically acceptable excipients and a therapeutically effective amount of a spiro-oxindole compound having the following formula (I):

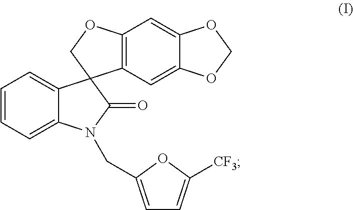

as a racemate, a single enantiomer, or a non-racemic mixture of enantiomers, or a pharmaceutically acceptable salt thereof, wherein the spiro-oxindole compound is present in a concentration of from about 0.1% w/w to about 25% w/w, wherein a first pharmaceutically acceptable excipient is Labrasol® and is present in a concentration of from about 35% w/w to about 65% w/w, wherein a second pharmaceutically acceptable excipient is Gelucire® 44/14 and is present in a concentration of from about 25% w/w to about 45% w/w, and wherein a third pharmaceutically acceptable excipient is propylene glycol and is present in a concentration of from about 1.0% w/w to about 10% w/w.

Another embodiment is a pharmaceutical composition for oral administration to a mammal comprising one or more pharmaceutically acceptable excipients and a therapeutically effective amount of a spiro-oxindole compound having the following formula (I):

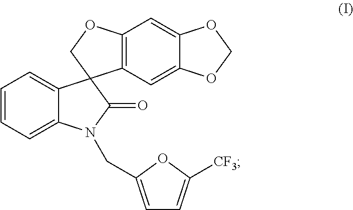

as a racemate, a single enantiomer, or a non-racemic mixture of enantiomers, or a pharmaceutically acceptable salt thereof, or a pharmaceutically acceptable salt thereof, wherein the spiro-oxindole compound is present in an unit dosage amount of between about 5 mg and about 100 mg, wherein a first pharmaceutically acceptable excipient is Labrasol® and is present in a concentration of from about 35% w/w to about 65% w/w, wherein a second pharmaceutically acceptable excipient is Gelucire® 44/14 and is present in a concentration of from about 25% w/w to about 45% w/w, and wherein a third pharmaceutically acceptable excipient is propylene glycol and is present in a concentration of from about 1.0% w/w to about 10% w/w.

Another embodiment is a pharmaceutical composition wherein the spiro-oxindole compound is the (S)-enantiomer of the compound of formula (I) having the following formula (I-S):

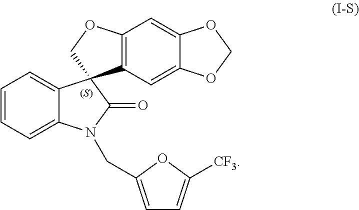

Another embodiment of the invention is a method of treating pain in a mammal, preferably a human, wherein the method comprises orally administering to the mammal in need thereof a therapeutically effective amount of a pharmaceutical composition of the invention, as set forth above in the Summary of the Invention, or a therapeutically effective amount of an embodiment of a pharmaceutical composition of the invention, as described above.

Another embodiment of the invention is a method of treating or lessening the severity of a disease, condition, or disorder where activation or hyperactivity of one or more of Naᵥ1.1, Naᵥ1.2, Naᵥ1.3, Naᵥ1.4, Naᵥ1.5, Naᵥ1.6, Naᵥ1.7, Naᵥ1.8, or Naᵥ1.9 is implicated in the disease state, wherein the method comprises orally administering to the mammal in need thereof a therapeutically effective amount of a pharmaceutical composition of the invention, as set forth above in the Summary of the Invention, or a therapeutically effective amount of an embodiment of a pharmaceutical composition of the invention, as described above.

Another embodiment of the invention is a method of treating a range of sodium channel-mediated diseases or conditions, including, but not limited to, pain of any nature, including but not limited to, pain associated with HIV, HIV treatment induced neuropathy, trigeminal neuralgia, post-herpetic neuralgia, diabetic neuropathy, peripheral neuropathy, Complex regional pain syndrome, Paroxysmal Extreme Pain Disorder, eudynia, familial erythromelalgia, secondary erythromelalgia, primary/inherited erythromelalgia, familial rectal pain, familial facial pain, dental pain, migraine, headache, familial hemiplegic migraine, sinus headache, tension headache, phantom limb pain, peripheral nerve injury, pain associated with multiple sclerosis (MS); myasthenia syndromes, myotonia, paroxysmal dystonia, periodic paralysis, spasticity, spastic paraplegia, myopathies, paramyotonia congentia, hyperkalemic periodic paralysis, hypokalemic periodic paralysis, malignant hyperthermia, heat sensitivity, irritable bowel syndrome, Crohns disease, motor impairment associated with MS, amyotrophic lateral sclerosis (ALS), pruritis, benign prostatic hyperplasia, arthritis, rheumatoid arthritis, osteoarthritis, cystic fibrosis, pseudoaldosteronism, rhabdomyolysis, bipolar depression, anxiety, schizophrenia, illness due to exposure to insecticides or other sodium channel toxins, cancer, epilepsy, partial and general tonic seizures, restless leg syndrome, fibromyalgia, neuroprotection under ischaemic conditions caused by stroke, glaucoma or neural trauma, arrhythmias, long-QT syndrome, Catecholeminergic polymorphic ventricular tachycardia, tachy-arrhythmias, atrial fibrillation and ventricular fibrillation, wherein the method comprises orally administering to the mammal in need thereof a therapeutically effective amount of a pharmaceutical composition of the invention, as set forth above in the Summary of the Invention, or a therapeutically effective amount of an embodiment of a pharmaceutical composition of the invention, as described above.

Another embodiment of the invention is a method of treating a range of sodium channel-mediated disease or condition through inhibition of ion flux through a voltage-dependent sodium channel in a mammal, preferably a human, wherein the method comprises orally administering to the mammal in need thereof a therapeutically effective amount of a pharmaceutical composition of the invention, as set forth above in the Summary of the Invention, or a therapeutically effective amount of an embodiment of a pharmaceutical composition of the invention, as described above.

Another embodiment of the invention is a method of treating or preventing benign prostatic hyperplasia in a mammal, preferably a human, wherein the method comprises orally administering to the mammal in need thereof a therapeutically effective amount of a pharmaceutical composition of the invention, as set forth above in the Summary of the Invention, or a therapeutically effective amount of an embodiment of a pharmaceutical composition of the invention, as described above.

Another embodiment of the invention is a method of treating or preventing pruritis in a mammal, preferably a human, wherein the method comprises orally administering to the mammal in need thereof a therapeutically effective amount of a pharmaceutical composition of the invention, as set forth above in the Summary of the Invention, or a therapeutically effective amount of an embodiment of a pharmaceutical composition of the invention, as described above.

Another embodiment of the invention is a method of treating or preventing cancer in a mammal, preferably a human, wherein the method comprises orally administering to the mammal in need thereof a therapeutically effective amount of a pharmaceutical composition of the invention, as set forth above in the Summary of the Invention, or a therapeutically effective amount of an embodiment of a pharmaceutical composition of the invention, as described above.

Specific embodiments of the pharmaceutical compositions of the invention and methods of using the pharmaceutical compositions of the invention are described in more detail below.

### Utility of the Pharmaceutical Compositions of the Invention

The present invention relates to pharmaceutical compositions and methods of using the pharmaceutical compositions comprising a therapeutically effective amount of a spiro-oxindole compound of the invention, as a racemate, a single enantiomer, or a non-racemic mixture of enantiomers, or a pharmaceutically acceptable salt thereof, for the treatment of sodium channel-mediated diseases, including, but not limited to, pain of any nature, including but not limited to, pain associated with HIV, HIV treatment induced neuropathy, trigeminal neuralgia, post-herpetic neuralgia, diabetic neuropathy, peripheral neuropathy, Complex regional pain syndrome, Paroxysmal Extreme Pain Disorder, eudynia, familial erythromelalgia, secondary erythromelalgia, primary/inherited erythromelalgia, familial rectal pain, familial facial pain, dental pain, migraine, headache, familial hemiplegic migraine, sinus headache, tension headache, phantom limb pain, peripheral nerve injury, pain associated with multiple sclerosis (MS); myasthenia syndromes, myotonia, paroxysmal dystonia, periodic paralysis, spasticity, spastic paraplegia, myopathies, paramyotonia congentia, hyperkalemic periodic paralysis, hypokalemic periodic paralysis, malignant hyperthermia, heat sensitivity, irritable bowel syndrome, Crohns disease, motor impairment associated with MS, amyotrophic lateral sclerosis (ALS), pruritis, benign prostatic hyperplasia, arthritis, rheumatoid arthritis, osteoarthritis, cystic fibrosis, pseudoaldosteronism, rhabdomyolysis, bipolar depression, anxiety, schizophrenia, illness due to exposure to insecticides or other sodium channel toxins, cancer, epilepsy, partial and general tonic seizures, restless leg syndrome, fibromyalgia, neuroprotection under ischaemic conditions caused by stroke, glaucoma or neural trauma, arrhythmias, long-QT syndrome, Catecholeminergic polymorphic ventricular tachycardia, tachy-arrhythmias, atrial fibrillation and ventricular fibrillation, by orally administering to a mammal, preferably a human in need thereof, a therapeutically effective amount of a pharmaceutical composition of the invention.

The general value of a spiro-oxindole compound of the invention, as a racemate, a single enantiomer, or a non-racemic mixture of enantiomers, or a pharmaceutically acceptable salt thereof, used in the pharmaceutical compositions of the invention in mediating, especially inhibiting, the sodium channel ion flux has been determined using the assays described in PCT Published Patent Application No. WO 06/110917. The general value of a spiro-oxindole compound of the invention, as a racemate, a single enantiomer, or a non-racemic mixture of enantiomers, or a pharmaceutically acceptable salt thereof, used in the pharmaceutical compositions of the invention in treating sodium-channel mediated diseases or conditions may be established in industry standard animal models and the animals disclosed in PCT Published Patent Application No. WO 06/110917 for demonstrating the efficacy of a spiro-oxindole compound of the invention, as a racemate, a single enantiomer, or a non-racemic mixture of enantiomers, or a pharmaceutically acceptable salt thereof, in treating such diseases and conditions.

As defined herein, a sodium channel-mediated disease or condition refers to a disease or condition which is ameliorated upon modulation of the sodium channel and includes, but is not limited to, pain of any nature, including but not limited to, pain associated with HIV, HIV treatment induced neuropathy, trigeminal neuralgia, post-herpetic neuralgia, diabetic neuropathy, peripheral neuropathy, Complex regional pain syndrome, Paroxysmal Extreme Pain Disorder, eudynia, familial erythromelalgia, secondary erythromelalgia, primary/inherited erythromelalgia, familial rectal pain, familial facial pain, dental pain, migraine, headache, familial hemiplegic migraine, sinus headache, tension headache, phantom limb pain, peripheral nerve injury, pain associated with multiple sclerosis (MS); myasthenia syndromes, myotonia, paroxysmal dystonia, periodic paralysis, spasticity, spastic paraplegia, myopathies, paramyotonia congentia, hyperkalemic periodic paralysis, hypokalemic periodic paralysis, malignant hyperthermia, heat sensitivity, irritable bowel syndrome, Crohns disease, motor impairment associated with MS, amyotrophic lateral sclerosis (ALS), pruritis, benign prostatic hyperplasia, arthritis, rheumatoid arthritis, osteoarthritis, cystic fibrosis, pseudoaldosteronism, rhabdomyolysis, bipolar depression, anxiety, schizophrenia, illness due to exposure to insecticides or other sodium channel toxins, cancer, epilepsy, partial and general tonic seizures, restless leg syndrome, fibromyalgia, neuroprotection under ischaemic conditions caused by stroke, glaucoma or neural trauma, arrhythmias, long-QT syndrome, Catecholeminergic polymorphic ventricular tachycardia, tachy-arrhythmias, atrial fibrillation and ventricular fibrillation.

As used herein, the term “pain” refers to all categories of pain and is recognized to include, but is not limited to, neuropathic pain, inflammatory pain, nociceptive pain, idiopathic pain, neuralgic pain, orofacial pain, burn pain, burning mouth syndrome, somatic pain, visceral pain, myofacial pain, dental pain, cancer pain, chemotherapy pain, trauma pain, surgical pain, post-surgical pain, childbirth pain, labor pain, reflex sympathetic dystrophy, brachial plexus avulsion, neurogenic bladder, acute pain (e.g. musculoskeletal and post-operative pain), chronic pain, persistent pain, peripherally mediated pain, centrally mediated pain, chronic headache, migraine headache, familial hemiplegic migraine, conditions associated with cephalic pain, sinus headache, tension headache, phantom limb pain, peripheral nerve injury, pain following stroke, thalamic lesions, radiculopathy, HIV pain, post-herpetic pain, non-cardiac chest pain, irritable bowel syndrome and pain associated with bowel disorders and dyspepsia, and combinations thereof.

A spiro-oxindole compound of the invention, as a racemate, a single enantiomer, or a non-racemic mixture of enantiomers, or a pharmaceutically accceptable salt thereof, utilized in the pharmaceutical compositions of the invention is also useful in treating or preventing other disorders such as benign prostatic hyperplasia (BPH) and pruritis (itch).

Benign prostatic hyperplasia (BPH), also known as benign prostatic hypertrophy, is one of the most common diseases affecting aging men. BPH is a progressive condition which is characterized by a nodular enlargement of prostatic tissue resulting in obstruction of the urethra. Consequences of BPH can include hypertrophy of bladder smooth muscle, a decompensated bladder, acute urinary retention and an increased incidence of urinary tract infection.

BPH has a high public health impact and is one of the most common reasons for surgical intervention among elderly men. Attempts have been made to clarify the etiology and pathogenesis and, to that end, experimental models have been developed. Spontaneous animal models are limited to the chimpanzee and the dog. BPH in man and the dog share many common features. In both species, the development of BPH occurs spontaneously with advanced age and can be prevented by early/prepubertal castration. A medical alternative to surgery is very desirable for treating BPH and the consequences.

The prostatic epithelial hyperplasia in both man and the dog is androgen sensitive, undergoing involution with androgen deprivation and resuming epithelial hyperplasia when androgen is replaced. Cells originating from the prostate gland have been shown to express high levels of voltage gated sodium channels. Immunostaining studies clearly demonstrated evidence for voltage gated sodium channels in prostatic tissues (Prostate Cancer Prostatic Dis. 2005; 8(3):266-73).

Pruritis, commonly known as itch, is a common dermatological condition. While the exact causes of pruritis are complex and poorly understood, there has long been acknowledged to have interactions with pain. In particular, it is believed that sodium channels likely communicate or propagate along the nerve axon the itch signals along the skin. Transmission of the itch impulses results in the unpleasant sensation that elicits the desire or reflex to scratch.

From a neurobiology level, it is believed that there is a shared complexity of specific mediators, related neuronal pathways and the central processes of itch and pain and recent data suggest that there is a broad overlap between pain- and itch-related peripheral mediators and/or receptors (Ikoma et al., Nature Reviews Neuroscience, 7:535-547, 2006). Remarkably, pain and itch have similar mechanisms of neuronal sensitization in the peripheral nervous system and the central nervous system but exhibits intriguing differences as well.

For example, the mildly painful stimuli from scratching are effective in abolishing the itch sensation. In contrast, analgesics such as opioids can generate severe pruritis. The antagonistic interaction between pain and itch can be exploited in pruritis therapy, and current research concentrates on the identification of common targets for future analgesic and antipruritic therapy. A spiro-oxindole compound of the invention, as a racemate, a single enantiomer, or a non-racemic mixture of enantiomers, or a pharmaceutically acceptable salt thereof, has been shown to have analgesic effects in a number of animal models at oral doses ranging from 1 mg/Kg to 100 mg/Kg. Accordingly, a spiro-oxindole compound of the invention, as a racemate, a single enantiomer, or a non-racemic mixture of enantiomers, or a pharmaceutically acceptable salt thereof, can also be useful for treating pruritis.

The types of itch or skin irritation, include, but are not limited to:

a) psoriatic pruritis, itch due to hemodyalisis, aguagenic pruritis, and itching caused by skin disorders (e.g., contact dermatitis), systemic disorders, neuropathy, psychogenic factors or a mixture thereof;

b) itch caused by allergic reactions, insect bites, hypersensitivity (e.g., dry skin, acne, eczema, psoriasis), inflammatory conditions or injury;

c) itch associated with vulvar vestibulitis; and

d) skin irritation or inflammatory effect from administration of another therapeutic such as, for example, antibiotics, antivirals and antihistamines; and

e) itch due to activation of PAR-2 G-protein coupled receptors.

A spiro-oxindole compound of the invention, as a racemate, a single enantiomer, or a non-racemic mixture of enantiomers, or a pharmaceutically acceptable salt thereof, utilized in the pharmaceutical compositions of the invention is also useful in treating or preventing certain hormone sensitive cancers, such as prostate cancer (adenocarcinoma), breast cancer, ovarian cancer, testicular cancer, and thyroid neoplasia. The voltage gated sodium channels have been demonstrated to be expressed in prostate and breast cancer cells. Up-regulation of neonatal Naᵥ1.5 occurs as an integral part of the metastatic process in human breast cancer and could serve both as a novel marker of the metastatic phenotype and a therapeutic target (Clin. Cancer Res. 2005, Aug. 1; 11(15): 5381-9). Functional expression of voltage-gated sodium channel alpha-subunits, specifically Naᵥ1.7, is associated with strong metastatic potential in prostate cancer (CaP) in vitro. Voltage-gated sodium channel alpha-subunits immunostaining, using antibodies specific to the sodium channel alpha subunit was evident in prostatic tissues and markedly stronger in CaP vs non-CaP patients (Prostate Cancer Prostatic Dis. 2005; 8(3):266-73)

A spiro-oxindole compound of the invention, as a racemate, a single enantiomer, or a non-racemic mixture of enantiomers, or a pharmaceutically acceptable salt thereof, utilized in the pharmaceutical compositions of the invention is also useful in treating or preventing symptoms associated with BPH such as, but not limited to, acute urinary retention and urinary tract infection.

A spiro-oxindole compound of the invention, as a racemate, a single enantiomer, or a non-racemic mixture of enantiomers, or a pharmaceutically acceptable salt thereof, utilized in the pharmaceutical compositions of the invention is also useful in treating or preventing certain endocrine imbalances or endocrinopathies such as congenital adrenal hyperplasia, hyperthyroidism, hypothyroidism, osteoporosis, osteomalacia, rickets, Cushing's Syndrome, Conn's syndrome, hyperaldosteronism, hypogonadism, hypergonadism, infertility, fertility and diabetes.

Accordingly, pharmaceutical compositions of the invention comprising one or more pharmaceutically acceptable excipients and a therapeutically effective amount of a spiro-oxindole compound of the invention, as a racemate, a single enantiomer, or a non-racemic mixture of enantiomers, or a pharmaceutically acceptable salt thereof, are useful in treating the diseases and conditions set forth above.

### Preparation of the Pharmaceutical Compositions of the Invention

### Preparation of the Spiro-Oxindole Compounds of the Invention

The spiro-oxindole compounds of the invention can be prepared by the methods disclosed in PCT Published Patent Application No. WO 06/110917. The preparation of COMPOUND A, or a pharmaceutically acceptable salt thereof, is specifically disclosed in PCT Published Patent Application No. WO 06/110917.

COMPOUND B is prepared by the resolution of COMPOUND A, using either chiral high pressure liquid chromatography methods or by simulated moving bed chromatography methods, as described below in the following Reaction Scheme wherein “chiral HPLC” refers to chiral high pressure liquid chromatography and “SMB” refers to simulated moving bed chromatography:

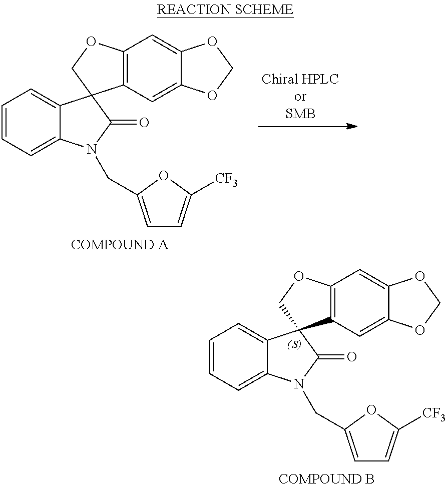

The following Synthetic Examples serve to illustrate the resolution methods disclosed by the above Reaction Scheme and are not intended to limit the scope of the invention.

### SYNTHETIC EXAMPLE 1

### Synthesis of 1′-{[5-(trifluoromethyl)furan-2-yl]methyl}spiro[furo[2,3-f][1,3]benzodioxole-7,3′-indol]-2′(1′H)-one (COMPOUND A)

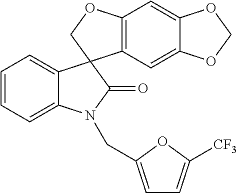

To a suspension of spiro[furo[2,3-f][1,3]benzodioxole-7,3′-indol]-2′(1′H)-one (1.0 g, 3.6 mmol), which can be prepared according to the methods disclosed in PCT Published Patent Application No. WO 2006/110917, and cesium carbonate (3.52 g, 11 mmol) in acetone (50 mL) was added 2-bromomethyl-5-trifluoromethylfuran (1.13 g, 3.9 mmol) in one portion and the reaction mixture was stirred at 55-60° C. for 16 hours. Upon cooling to ambient temperature, the reaction mixture was filtered and the filtrate was evaporated under reduced pressure. The residue was subjected to column chromatography, eluting with ethyl acetate/hexane (1/9-1/1) to afford 1′-{[5-(trifluoromethyl)furan-2-yl]methyl}spiro[furo[2,3-f][1,3]benzodioxole-7,3′-indol]-2′(1′H)-one, i.e., the compound of formula (I), (1.17 g, 76%) as a white solid: mp 139-141° C.; ¹H NMR (300 MHz, CDCl₃) δ 7.32-6.97 (m, 5H), 6.72 (d, J=3.24 Hz, 1H), 6.66 (s, 1H), 6.07 (s, 1H), 5.90-5.88 (m, 2H), 5.04 (ABq, 2H), 4.74 (ABq, 2H); ¹³C NMR (75 MHz, CDCl₃) δ 176.9, 155.7, 153.5, 148.8, 142.2, 141.9, 140.8, 140.2, 139.7, 139.1, 132.1, 129.2, 124.7, 124.1, 123.7, 121.1, 120.1, 117.6, 114.5, 114.4, 110.3, 109.7, 103.0, 101.9, 93.8, 80.0, 57.8, 36.9; MS (ES+) m/z 430.2 (M+1), 452.2 (M+23); Cal'd for C₂₂H₁₄F₃NO₅: C, 61.54%; H, 3.29%; N, 3.26%; Found: C, 61.51%; H, 3.29%; N, 3.26%.

### SYNTHETIC EXAMPLE 2

### Isolation of COMPOUND B by Chiral HPLC

COMPOUND B was isolated by resolving COMPOUND A under the following chiral HPLC conditions:
Column: Chiralcel® OJ-RH; 20 mm I.D.×250 mm, 5 mic; Lot: OJRH CJ-EH001 (Daicel Chemical Industries, Ltd)
Eluent: Acetonitrile/Water (60/40, v/v, isocratic)
Flow rate: 10 mL/min
Run time: 60 min
Loading: 100 mg of COMPOUND A in 1 mL of acetonitrile
Temperature: Ambient

Under the above chiral HPLC conditions, the (R)-enantiomer of COMPOUND A was isolated as the first fraction as a white solid. COMPOUND B was isolated as the second fraction as a white solid; ee>99% (analytical OJRH, 55% acetonitrile in water); mp 100-102° C.; ¹H NMR (300 MHz, DMSO-d₆) δ 7.32-6.99 (m, 5H), 6.71 (d, J=3.43 Hz, 1H), 6.67 (s, 1H), 6.05 (s, 1H), 5.89 (ABq, 2H), 5.03 (ABq, 2H), 4.73 (ABq, 2H); ¹³C NMR (75 MHz, CDCl₃) δ 177.2, 155.9, 152.0, 149.0, 142.4, 142.0, 141.3, 132.0, 129.1, 123.9, 120.6, 119.2, 117.0, 112.6, 109.3, 108.9, 103.0, 101.6, 93.5, 80.3, 58.2, 36.9; MS (ES+) m/z 430.2 (M+1), [α]_{D}+14.04 (c 0.99, DMSO).

### SYNTHETIC EXAMPLE 3

### Isolation of COMPOUND B by SMB Chromatography

COMPOUND B was isolated by resolving COMPOUND A under the following SMB chromatography conditions:
Extract: 147.05 mL/min
Raffinate: 86.13 mL/min
Eluent: 183.18 mL/min
Feed: 50 mL/min
Recycling: 407.88 mL/min
Run Time: 0.57 min
Temperature: 25° C.
Pressure: 55 bar

The feed solution (25 of COMPOUND A in 1.0 L of mobile phase (25:75 (v:v:v) mixture of acetonitrile/methanol)) was injected continuously into the SMB system (Novasep Licosep Lab Unit), which was equipped with eight identical columns in 2-2-2-2 configuration containing 110 (per column, 9.6 cm, 4.8 cm I.D.) of chiralpack AD as stationary phase. The first eluting enantiomer (the (R)-enantiomer of COMPOUND A) was contained in the raffinate stream and the second eluting enantiomer (COMPOUND B) was contained in the extract stream. The characterization data of COMPOUND B obtained from the SMB resolution were identical to those obtained above utilizing chiral HPLC.

COMPOUND A was resolved into its constituent enantiomers on a Waters preparative LCMS autopurification system. The first-eluting enantiomer from the chiral column was brominated (at a site well-removed from the stereogenic centre) to give the corresponding 5′-bromo derivative, which was subsequently crystallized to generate a single crystal suitable for X-ray crystallography. The crystal structure of this brominated derivative of the first-eluting enantiomer was obtained and its absolute configuration was found to be the same as the (R)-enantiomer of COMPOUND A. Hence, the second-eluting enantiomer from the chiral column is the (S)-enantiomer of COMPOUND A. Moreover, the material obtained from the extract stream of the SMB resolution had a specific optical rotation of the same sign (positive, i.e. dextrorotatory) as that of the material obtained from the aforementioned LC resolution.

### Preparation of the Pharmaceutical Compositions of the Invention

The preparation of the pharmaceutical compositions of the invention employs conventional techniques of pharmaceutical formulation, medicinal chemistry and the like, which are within the skill of the art. Such techniques are explained fully in the literature. Preparation of pharmaceutical compositions are described, for example, in Remington: The Science and Practice of Pharmacy, 21^{st} edition (Lippincott Williams & Wilkins, (2005) and Ansel et al., Pharmaceutical Dosage Forms and Drug Delivery Systems, 8^{th} Ed. (Med, P A: Williams & Wilkins, 2005).

In general, the pharmaceutical compositions of the invention can be prepared by combining a spiro-oxindole compound of the invention, as a racemate, a single enantiomer, or a non-racemic mixture of enantiomers, or a pharmaceutically acceptable salt thereof, with one or more pharmaceutically acceptable excipients. The pharmaceutical compositions of the invention may be formulated for oral administration into preparations in solid, semi-solid (gel), or liquid forms, such as tablets, liquid-filled capsules, gel-filled capsules, powders, granules, solutions, gels, and microspheres. Preferably, the pharmaceutical compositions are formulated in semi-solid (gel) or liquid form.

The pharmaceutical compositions of the invention may include various materials which modify the physical form of the pharmaceutical compositions. For example, the pharmaceutical compositions of the invention may be in solid, semi-solid (gel) or liquid form and may include materials that form a coating or shell around the pharmaceutical composition. The materials that form the coating or shell are typically inert, and may be selected from, for example, sugar, shellac, and other enteric coating agents. Such coated or shelled pharmaceutical compositions are considered to be within the scope of pharmaceutical compositions of the invention. Alternatively, the pharmaceutical compositions may be encased in a gelatin or hydroxypropylmethyl cellulose (HPMC) capsule. Such encapsulated pharmaceutical compositions are considered to be within the scope of pharmaceutical compositions of the invention. Preferably, the pharmaceutical compositions of the invention are encapsulated by either a gelatin or HPMC capsule

The pharmaceutical compositions of the invention in solid, semi-solid (gel) or liquid form may additionally include a complexing agent that binds to a spiro-oxindole compound of the invention, as a racemate, a single enantiomer, or a non-racemic mixture of enantiomers, or a pharmaceutically acceptable salt thereof, or may additionally include a clathrate that molecularly encapsulates the spiro-oxindole compound of the invention, as a racemate, a single enantiomer, or a non-racemic mixture of enantiomers, or a pharmaceutically acceptable salt thereof, thereby assisting in the solubility of the spiro-oxindole compound of the invention and/or the delivery of the spiro-oxindole compound of the invention to the intended in vivo site. Suitable agents that may act in these capacities include monoclonal or polyclonal antibodies, proteins, liposomes and clathrates, including cyclodextrins such as α-cyclodextrin, β-cyclodextrin, γ-cyclodextrin, or modified cyclodextrins, such as hydroxypropyl-β-cyclodextrin (“HP-β-CD”) (e.g., Keptose® HPB).

The pharmaceutical compositions of the invention comprise one or more pharmaceutically acceptable excipients, which include, but are not limited to, any solvent, adjuvant, bioavailability enhancer, carrier, glidant, sweetening agent, diluent, preservative, dye/colorant, flavor enhancer, surfactant, wetting agent, dispersing agent, suspending agent, stabilizer, isotonic agent, buffer and/or emulsifier approved by the United States Food and Drug Administration, Health Canada or the European Medicines Agency, as being acceptable for use in humans or domestic animals. Exemplary pharmaceutically acceptable excipients include, but are not limited to, the following:
acetylated glycerides (e.g., Myvacet® 9-45K);
benzyl alcohol;
benzyl benzoate;
caprylic/capric triglycerides (e.g., Neobee® M-5);
diethyleneglycol monoethyl ether (e.g., Transcutol®);
dimethylamine (“DMA”);
ethanol;
glucose (solution);
glyceryl caprylate/caprate and PEG-8 (polyethylene glycol) caprylate/caprate complex (e.g., Labrasol®);
caprylic/capric glycerides (e.g., Imwitor® 742);
propylene glycol dicaprylocaptate (e.g., Labrafac®);
caprylocaproyl macrogolglycerides (e.g., Labrasol®);
isopropyl alcohol;
macrogol-15 hydroxystearate (e.g., Solutol® HS15);
medium chain triglycerides (e.g., Miglyol® 810, Miglyol® 840 or Miglyol® 812);
sulfobutylether-β-cyclodextrin (e.g., Capitsol®);
peanut oil;
polyethylene glycol (“PEG”);
polyethylene glycol 400 (“PEG 400”) (e.g., Lutrol® E 400);
polyethylene glycol 6000;
polyethylene polyoxypropylene copolymer (e.g., Lutrol® F127);
polyglycolized glyceride (e.g., Gelucire® 44/14);
polyoxyl 35 castor oil (e.g., Cremophor® EL and Cremophor® ELP);
polyoxyl 40 hydrogenated castor oil (e.g, Cremophor® RH 40);
poly(vinylpyrrolidinone (“PVP”, e.g., Kollidon® K30 or Plasdone® K29/32);
polysorbate 80 (e.g., Tween® 80);
propylene glycol monocaprylate (e.g., Capmul® PG8);
propylene glycol monocaprylate 90% (e.g., Capryol® 90);
propylene glycol dicaprylate/dicaprate (e.g., Captex 200P);
soybean oil;
α-tocopherol polyethylene glycol succinate (“TPGS”); and
water.

Additional pharmaceutically acceptable excipients are disclosed herein.

In the preparation of pharmaceutical compositions of the invention, extensive studies were conducted to provide pharmaceutical compositions which allowed for the desired therapeutically effective amount of a spiro-oxindole compound of the invention, as a racemate, a single enantiomer, or a non-racemic mixture of enantiomers, or a pharmaceutically acceptable salt thereof, to be dissolved in one or more pharmaceutically acceptable excipients and which allowed for the spiro-oxindole compound of the invention, as a racemate, a single enantiomer, or a non-racemic mixture of enantiomers, or a pharmaceutically acceptable salt thereof, to be readily absorbed in a suitable period of time after oral administration (e.g., by ingestion). Furthermore, the pharmaceutical compositions needed to be stable over a suitable period of time.

The pharmaceutical compositions of the invention comprise a therapeutically effective amount of a spiro-oxindole compound of the invention, as a racemate, a single enantiomer, or a non-racemic mixture of enantiomers, or a pharmaceutically acceptable salt thereof. The spiro-oxindole compound of the invention, as a racemate, a single enantiomer, or a non-racemic mixture of enantiomers, or a pharmaceutically acceptable salt thereof, is an analgesic in development for the treatment of pain and more particularly for the treatment of chronic neuropathic and osteoarthritic pain. In humans the expected oral efficacy for the treatment of pain is between about 20 and about 200 mg/day, e.g. 50 mg, 100 mg or 200 mg per day. The spiro-oxindole compound of the invention, as a racemate, a single enantiomer, or a non-racemic mixture of enantiomers has very limited aqueous solubility (<5 μg/mL) and is a neutral compound. The spiro-oxindole compound of the invention, as a racemate, a single enantiomer, or a non-racemic mixture of enantiomers does not contain functional groups that can be ionised by pH alteration and consequently varying the pH of a solution to 2, 7.4 and 12 does not change the solubility of the spiro-oxindole compound of the invention, which remains at <5 μg/mL.

The following Table 1 lists excipients which were shown to be suitable for producing a stable solution of a spiro-oxindole compound of the invention, as a racemate, a single enantiomer, or a non-racemic mixture of enantiomers, or a pharmaceutically acceptable salt thereof, upon mixing, vortexing and/or heating to 70-80° C.:

TABLE 1
SUITABLE EXCIPIENTS
Trade Name

Capmul ® PG8
Capryol ® 90
Captex ® 200P
Corn Oil
Cremophor ® ELP
Imwitor ® 742
Labrafac ®
Labrasol ®
Miglyol ® 840
Myvacet ® 9-45K
Neobee ® M-5
PEG 400
PVP
Solutol ® HS 15
TPGS
Transcutol ®

Determination of the solubility of a spiro-oxindole compound of the invention, as a racemate, a single enantiomer, or a non-racemic mixture of enantiomers, or a pharmaceutically acceptable salt thereof, in each of the above excipients was carried out by weighing a specified quantity of the excipient into a scintillation vial and then adding a weighed quantity of the spiro-oxindole compound of the invention, as a racemate, a single enantiomer, or a non-racemic mixture of enantiomers, or a pharmaceutically acceptable salt thereof. Heating and vortexing was then applied as required to dissolve the spiro-oxindole compound of the invention, as a racemate, a single enantiomer, or a non-racemic mixture of enantiomers, or a pharmaceutically acceptable salt thereof. If the initial quantity of the spiro-oxindole compound of the invention, as a racemate, a single enantiomer, or a non-racemic mixture of enantiomers, or a pharmaceutically acceptable salt thereof, dissolved, then an additional amount of the spiro-oxindole compound of the invention, as a racemate, a single enantiomer, or a non-racemic mixture of enantiomers, or a pharmaceutically acceptable salt thereof, was added to determine the maximum solubility.

The following Table 2 lists combinations of excipients which were shown to be suitable for producing a solution of COMPOUND A, or a pharmaceutically acceptable salt thereof, upon mixing and heating to 70-80° C. The rationale for preparing each combination is given as well:

TABLE 2
SUITABLE EXCIPIENT COMBINATIONS

| EXCIPIENT COMBINATION | Rationale |
|---|---|
| PEG 400 + PEG 6000 + Cremophor ® RH40 | COMPOUND A was shown to be |
| | readily soluble in PEG 400. PEG |
| | 6000 was utilized to increase |
| | compatibility of the combination with |
| | gelatin capsules at 100% |
| | concentration. The excipient |
| | Cremophor ® RH40 was included as a |
| | surfactant. |
| Peg 400 + PEG 6000 + Labrasol ® | Same rationale as above except that |
| | the Cremophor ® RH40 was replaced |
| | with the excipient Labrasol ®. |
| Labrafac ® + Cremophor ® RH40 | COMPOUND A was shown to be |
| | readily soluble in Labrafac ®. The |
| | excipient Cremophor ® RH40 was |
| | included as a surfactant. |
| Labrasol ® + Labrafac ® + Tween ® 80 | COMPOUND A was shown to be |
| | soluble in both Labrasol ® and |
| | Labrafac ®, and the exicipient Tween ® |
| | 80 was included as surfactant. |
| Labrasol ® + Gelucire ® + Propylene Glycol | COMPOUND A was shown to be |
| | soluble in Labrasol ®, the excipient |
| | Gelucire ® was included as a |
| | bioavailability enhancer and the |
| | excipient propylene glycol (a |
| | hydrophilic co-solvent) was included |
| | to increase solvent capacity. |
| Labrasol ® + Capryol ® 90 | Combination of an excipient with high |
| | hydrophile-lipophile balance (HLB) |
| | (Labrasol ®) and an excipient with a |
| | low HLB (Capryol 90 ®). |
| Labrasol ® + Solutol ® | Combination of an excipient with high |
| | HLB (Labrasol ®) and an excipient |
| | with a low HLB (Solutol ®). |
| PEG 6000 + PEG 400 + Lutrol ® F127 | Combination of PEG 400 and PEG |
| | 6000 with the excipient Lutrol ® F127 |
| | included as a surfactant. |

Based on the results of these solubility studies, and noting that Labrasol® was the best excipient with respect to the solubility of COMPOUND A, or a pharmaceutically acceptable salt thereof, therein which had been previously used in humans, the following pharmaceutical compositions of the invention were prepared in liquid form for dissolution and pharmacokinetic studies. These compositions were prepared by adding the indicated excipients into a 250 mL stainless steel container on a hot plate equipped with a magnetic stirrer. The excipients were then heated to 70-80° C. with stirring. Once heated, the desired quantity of the active ingredient, was added and stirring of the resulting solution was continued until the active ingredient was dissolved. The heat was then reduced to 60-65° C. and the desired weight of the resulting solution was hand filled into hard gelatin capsules (Licaps®) using a Micromans® pipette. The filled capsules were then manually closed.

Accordingly, in the following pharmaceutical compositions of the invention, “COMPOUND A” is intended to include COMPOUND A and pharmaceutically acceptable salts of COMPOUND A.

COMPOUND A 100 MG COMPOSITION #1

| | Amount | Conc. |
|---|---|---|
| Ingredient | mg/capsule | % w/w |
| COMPOUND A | 100 | 18.2 |
| Labrasol ® | 250 | 45.5 |
| PEG 400 | 100 | 18.2 |
| PEG 6000 | 100 | 18.2 |
| Total mg/capsule | 550 mg | |

COMPOUND A 100 MG COMPOSITION #2

| | Amount | Conc. |
|---|---|---|
| Ingredient | mg/capsule | % w/w |
| COMPOUND A | 100 | 14.3 |
| Labrasol ® | 300 | 42.9 |
| Capryol ® 90 | 300 | 42.9 |
| Total mg/capsule | 700 mg | |

COMPOUND A 100 MG COMPOSITION #3

| | Amount | Conc. |
|---|---|---|
| Ingredient | mg/capsule | % w/w |
| COMPOUND A | 100 | 15.4 |
| Labrasol ® | 250 | 38.5 |
| Gelucire ® 44/14 | 250 | 38.5 |
| Propylene Glycol | 50 | 7.7 |
| Total mg/capsule | 650 mg | |

COMPOUND A 100 MG COMPOSITION #4

| | Amount | Conc. |
|---|---|---|
| Ingredient | mg/capsule | % w/w |
| COMPOUND A | 100 | 16.7 |
| Labrasol ® | 250 | 41.7 |
| Gelucire ® 44/14 | 250 | 41.7 |
| Total mg/capsule | 600 mg | |

COMPOUND A 100 MG COMPOSITION #5

| | Amount | Conc. |
|---|---|---|
| Ingredient | mg/capsule | % w/w |
| COMPOUND A | 100 | 16.7 |
| Labrasol ® | 225 | 37.5 |
| Gelucire ® 44/14 | 225 | 37.5 |
| PEG 400 | 50 | 8.3 |
| Total mg/capsule | 600 mg | |

COMPOUND A 100 MG COMPOSITION #6

| | Amount | Conc. |
|---|---|---|
| Ingredient | mg/capsule | % w/w |
| COMPOUND A | 100 | 16.7 |
| Labrasol ® | 300 | 50.0 |
| Gelucire ® 44/14 | 150 | 25.0 |
| Propylene Glycol | 50 | 8.3 |
| Total mg/capsule | 600 mg | |

COMPOUND A 100 MG COMPOSITION #7

| | Amount | Conc. |
|---|---|---|
| Ingredient | mg/capsule | % w/w |
| COMPOUND A | 100 | 16.7 |
| Labrasol ® | 250 | 41.7 |
| Gelucire ® 44/14 | 200 | 33.3 |
| Propylene Glycol | 50 | 8.3 |
| Total mg/capsule | 600 mg | |

COMPOUND A 100 MG COMPOSITION #8

| | Amount | Conc. |
|---|---|---|
| Ingredient | mg/capsule | % w/w |
| COMPOUND A | 100 | 16.7 |
| Labrasol ® | 375 | 62.5 |
| PEG 400 | 25 | 4.2 |
| PEG 6000 | 100 | 16.7 |
| Total mg/capsule | 600 mg | |

A well known classification of lipid formulations may be found in Pouton, C., Eur. J. Pharm. Sci. (2000), Vol. 11, No. 2, pp. S93-S98) wherein lipid formulations are disclosed as being grouped by size of their microemulsion and whether digestion plays a role in absorption and deposition with respect to the different groups. A Type III system is disclosed therein as being the smallest droplet-sized microemulsion that has a digestion component. A small droplet size leads to optimal physical stability of the emulsion. Accordingly, to achieve a Type III system, pharmaceutical compositions of the invention were prepared with a glyceride component of less than 20%, preferably around 10%, taking into account that the solubility of COMPOUND B, for example, in a caprylic/capric glycerides such as Imwitor® 742 is less than other excipients.

In the following pharmaceutical compositions of the invention, “COMPOUND B” is intended to include COMPOUND B and pharmaceutically acceptable salts of COMPOUND B.

COMPOUND B 40 MG COMPOSITION #1

| | Amount | Conc. |
|---|---|---|
| Ingredient | mg/capsule | % w/w |
| COMPOUND B | 40 | 8.0 |
| Cremophor ® ELP | 282 | 56.4 |
| Labrasol ® | 122.5 | 24.5 |
| Imwitor ® 742 | 45 | 9.0 |
| Vitamin E | 10.5 | 2.1 |
| Total mg/capsule | 500 mg | |

COMPOUND B 40 MG COMPOSITION #2

| | Amount | Conc. |
|---|---|---|
| Ingredient | mg/capsule | % w/w |
| COMPOUND B | 40 | 8.0 |
| Cremophor ® ELP | 202.5 | 40.5 |
| Labrasol ® | 202.5 | 40.5 |
| Imwitor ® 742 | 45 | 9.0 |
| Vitamin E | 10.5 | 2.1 |
| Total mg/capsule | 500 mg | |

COMPOUND B 40 MG COMPOSITION #3

| | Amount | Conc. |
|---|---|---|
| Ingredient | mg/capsule | % w/w |
| COMPOUND B | 40 | 8.0 |
| Cremophor ® ELP | 98.5 | 19.7 |
| Labrasol ® | 306 | 61.2 |
| Imwitor ® 742 | 45 | 9.0 |
| Vitamin E | 10.5 | 2.1 |
| Total mg/capsule | 500 mg | |

COMPOUND B 40 MG COMPOSITION #4

| | Amount | Conc. |
|---|---|---|
| Ingredient | mg/capsule | % w/w |
| COMPOUND B | 40 | 8.0 |
| Cremophor ® ELP | 287 | 57.4 |
| Labrasol ® | 92.5 | 18.5 |
| Imwitor ® 742 | 45 | 9.0 |
| Vitamin E | 10.5 | 2.1 |
| Kollidon ® K30 | 25 | 5.0 |
| Total mg/capsule | 500 mg | |

COMPOUND B 40 MG COMPOSITION #5

| | Amount | Conc. |
|---|---|---|
| Ingredient | mg/capsule | % w/w |
| COMPOUND B | 40 | 8.0 |
| Cremophor ® ELP | 92.5 | 18.5 |
| Labrasol ® | 287 | 57.4 |
| Imwitor ® 742 | 45 | 9.0 |
| Vitamin E | 10.5 | 2.1 |
| Kollidon ® K30 | 25 | 5.0 |
| Total mg/capsule | 500 mg | |

COMPOUND B 40 MG COMPOSITION #6

| | Amount | Conc. |
|---|---|---|
| Ingredient | mg/capsule | % w/w |
| COMPOUND B | 40 | 10.0 |
| Cremophor ® ELP | 220 | 55.0 |
| Labrasol ® | 96 | 23.9 |
| Imwitor ® 742 | 36 | 9.0 |
| Vitamin E | 8 | 2.1 |
| Total mg/capsule | 400 mg | |

COMPOUND B 40 MG COMPOSITION #7

| | Amount | Conc. |
|---|---|---|
| Ingredient | mg/capsule | % w/w |
| COMPOUND B | 40 | 10.0 |
| Cremophor ® ELP | 158 | 39.5 |
| Labrasol ® | 158 | 39.5 |
| Imwitor ® 742 | 36 | 9.0 |
| Vitamin E | 8.4 | 2.1 |
| Total mg/capsule | 400 mg | |

COMPOUND B 40 MG COMPOSITION #8

| | Amount | Conc. |
|---|---|---|
| Ingredient | mg/capsule | % w/w |
| COMPOUND B | 40 | 10.0 |
| Cremophor ® ELP | 77 | 19.2 |
| Labrasol ® | 239 | 59.7 |
| Imwitor ® 742 | 36 | 9.0 |
| Vitamin E | 8.4 | 2.1 |
| Total mg/capsule | 400 mg | |

COMPOUND B 40 MG COMPOSITION #9

| | Amount | Conc. |
|---|---|---|
| Ingredient | mg/capsule | % w/w |
| COMPOUND B | 40 | 10.0 |
| Cremophor ® ELP | 224 | 55.9 |
| Labrasol ® | 72 | 18.0 |
| Imwitor ® 742 | 36 | 9.0 |
| Vitamin E | 8.4 | 2.1 |
| Kollidon ® K30 | 20 | 5.0 |
| Total mg/capsule | 400 mg | |

COMPOUND B 40 MG COMPOSITION #10

| | Amount | Conc. |
|---|---|---|
| Ingredient | mg/capsule | % w/w |
| COMPOUND B | 40 | 10.0 |
| Cremophor ® ELP | 72 | 18.0 |
| Labrasol ® | 224 | 55.9 |
| Imwitor ® 742 | 36 | 9.0 |
| Vitamin E | 8.4 | 2.1 |
| Kollidon ® K30 | 20 | 5.0 |
| Total mg/capsule | 400 mg | |

COMPOUND B 40 MG COMPOSITION #11

| | Amount | Conc. |
|---|---|---|
| Ingredient | mg/capsule | % w/w |
| COMPOUND B | 40 | 12.0 |
| Cremophor ® ELP | 178 | 53.6 |
| Labrasol ® | 78 | 23.3 |
| Imwitor ® 742 | 30 | 9.0 |
| Vitamin E | 7 | 2.1 |
| Total mg/capsule | 333 mg | |

COMPOUND B 40 MG COMPOSITION #12

| | Amount | Conc. |
|---|---|---|
| Ingredient | mg/capsule | % w/w |
| COMPOUND B | 40 | 12.0 |
| Cremophor ® ELP | 128 | 38.5 |
| Labrasol ® | 128 | 38.5 |
| Imwitor ® 742 | 30 | 9.0 |
| Vitamin E | 7 | 2.1 |
| Total mg/capsule | 333 mg | |

COMPOUND B 40 MG COMPOSITION #13

| | Amount | Conc. |
|---|---|---|
| Ingredient | mg/capsule | % w/w |
| COMPOUND B | 40 | 12.0 |
| Cremophor ® ELP | 63 | 18.8 |
| Labrasol ® | 193 | 58.1 |
| Imwitor ® 742 | 30 | 9.0 |
| Vitamin E | 7 | 2.1 |
| Total mg/capsule | 333 mg | |

COMPOUND B 40 MG COMPOSITION #14

| | Amount | Conc. |
|---|---|---|
| Ingredient | mg/capsule | % w/w |
| COMPOUND B | 40 | 12.0 |
| Cremophor ® ELP | 181 | 54.4 |
| Labrasol ® | 58 | 17.5 |
| Imwitor ® 742 | 30 | 9.0 |
| Vitamin E | 7 | 2.1 |
| Kollidon ® K30 | 17 | 5.0 |
| Total mg/capsule | 333 mg | |

COMPOUND B 40 MG COMPOSITION #15

| | Amount | Conc. |
|---|---|---|
| Ingredient | mg/capsule | % w/w |
| COMPOUND B | 40 | 12.0 |
| Cremophor ® ELP | 58 | 17.5 |
| Labrasol ® | 181 | 54.4 |
| Imwitor ® 742 | 30 | 9.0 |
| Vitamin E | 7 | 2.1 |
| Kollidon ® K30 | 17 | 5.0 |
| Total mg/capsule | 333 mg | |

COMPOUND B 40 MG COMPOSITION #16

| | Amount | Conc. |
|---|---|---|
| Ingredient | mg/capsule | % w/w |
| COMPOUND B | 40 | 16.4 |
| Cremophor ® ELP | 134 | 55.0 |
| Labrasol ® | 43 | 17.7 |
| Imwitor ® 742 | 21 | 8.8 |
| Vitamin E | 5 | 2.1 |
| Total mg/capsule | 244 mg | |

COMPOUND B 40 MG COMPOSITION #17

| | Amount | Conc. |
|---|---|---|
| Ingredient | mg/capsule | % w/w |
| COMPOUND B | 40 | 4.9 |
| Cremophor ® ELP | 511 | 62.6 |
| Labrasol ® | 164 | 20.1 |
| Imwitor ® 742 | 82 | 10.0 |
| Vitamin E | 20 | 2.4 |
| Total mg/capsule | 816 mg | |

COMPOUND B 40 MG COMPOSITION #18

| | Amount | Conc. |
|---|---|---|
| Ingredient | mg/capsule | % w/w |
| COMPOUND B | 40 | 5.0 |
| Gelucire ® 44/14 | 304 | 38.0 |
| Labrasol ® | 380 | 47.5 |
| Propylene Glycol | 76 | 9.5 |
| Total mg/capsule | 800 mg | |

Based on the results of the dissolution and pharmacokinetic studies on the above compositions, COMPOUND A 100 mg Composition #7, as set forth above, was further prepared in 25 mg/capsule, 15 mg/capsule, 10 mg/capsule and 5 mg/capsule, as set forth below:

COMPOUND A 25 MG COMPOSITION #7-1

| | Amount | Conc. |
|---|---|---|
| Ingredient | mg/capsule | % w/w |
| COMPOUND A | 25 | 4.76 |
| Labrasol ® | 250 | 47.6 |
| Gelucire ® 44/14 | 200 | 38.1 |
| Propylene Glycol | 50 | 9.52 |
| Total mg/capsule | 525 mg | |

COMPOUND A 15 MG COMPOSITION #7-2

| | Amount | Conc. |
|---|---|---|
| Ingredient | mg/capsule | % w/w |
| COMPOUND A | 15 | 2.91 |
| Labrasol ® | 250 | 48.5 |
| Gelucire ® 44/14 | 200 | 38.8 |
| Propylene Glycol | 50 | 9.70 |
| Total mg/capsule | 515 mg | |

COMPOUND A 10 MG COMPOSITION #7-3

| | Amount | Conc. |
|---|---|---|
| Ingredient | mg/capsule | % w/w |
| COMPOUND A | 10 | 1.96 |
| Labrasol ® | 250 | 49.0 |
| Gelucire ® 44/14 | 200 | 39.2 |
| Propylene Glycol | 50 | 9.80 |
| Total mg/capsule | 510 mg | |

COMPOUND A 5 MG COMPOSITION #7-4

| | Amount | Conc. |
|---|---|---|
| Ingredient | mg/capsule | % w/w |
| COMPOUND A | 5 | 0.99 |
| Labrasol ® | 250 | 49.5 |
| Gelucire ® 44/14 | 200 | 39.6 |
| Propylene Glycol | 50 | 9.90 |
| Total mg/capsule | 505 mg | |

The total amount of a particular pharmaceutically acceptable excipient in a pharmaceutical composition of the invention for oral administration to a mammal, preferably a human, should not exceed the Acceptable Daily Intake (ADI) of the particular pharmaceutically acceptable excipient. In general, each pharmaceutically acceptable excipient may be present in a pharmaceutical composition of the invention in a concentration of from about 0.5% w/w to about 99.0% w/w. More preferred, each pharmaceutically acceptable excipient may be present in a pharmaceutical composition of the invention in a concentration of from about 1% w/w to about 90% w/w. Even more preferred, each pharmaceutically acceptable excipient may be present in a pharmaceutical composition of the invention in a concentration of from about 10% w/w to about 80.0% w/w.

The stability of the pharmaceutical compositions disclosed herein may be tested in convention manner, e.g., by measurement of the spiro-oxindole compound of the invention, as a racemate, a single enantiomer, or a non-racemic mixture of enantiomers, or a pharmaceutically acceptable salt thereof, and its degradation products, dissolution, friability, disintegration time, microbial content, appearance and/or microscopy, for defined periods of time.

Preferably, the pharmaceutical compositions of this invention will be stable for at least 6 or 12 months when kept at a temperature of 5 to 50° C. More preferably, they will be stable for at least 6 or 12 months when kept at a temperature of 15 to 45° C. Most preferably, they will be stable for at least 6 to 12 months when kept at a temperature of 25 to 40° C. In a more preferred embodiment, the pharmaceutical compositions are stable over a period of time such as a year, and preferably 2 years. More preferably, the pharmaceutical compositions are stable for 3 years.

Accordingly, in one embodiment of the invention, a process for the preparation of a capsule containing a pharmaceutical composition of the invention for oral administration to a mammal, preferably a human, wherein the pharmaceutical composition comprises a therapeutically effective amount of a spiro-oxindole compound of the invention, as a racemate, a single enantiomer, or a non-racemic mixture of enantiomers, or a pharmaceutically acceptable salt thereof, and one or more pharmaceutically effective excipients, is performed by dissolving the desired therapeutically effective amount of the spiro-oxindole compound of the invention, as a racemate, a single enantiomer, or a non-racemic mixture of enantiomers, or a pharmaceutically acceptable salt thereof, preferably COMPOUND A, or a pharmaceutically acceptable salt thereof, in Labrosol® in the amounts listed below in Table 3 at preferably 65 to 85° C. with the addition of Gelucire® 44/14 and propylene glycol. The resultant solution is mixed for a suitable period of time, preferably for a period of time of between about 30 minutes and about 1 hour. Upon completion (when the spiro-oxindole compound of the invention, as a racemate, a single enantiomer, or a non-racemic mixture of enantiomers, or a pharmaceutically acceptable salt thereof, is completely dissolved), the resultant solution is gradually cooled down to a suitable temperature, preferably to a temperature of between about 30° C. and about 40° C. and transferred into capsule-filling equipment. Capsules capable of containing the requisite volume of the pharmaceutical composition so prepared in order to administer a therapeutically effective amount of a spiro-oxindole compound of the invention, as a racemate, a single enantiomer, or a non-racemic mixture of enantiomers, or a pharmaceutically acceptable salt thereof, are then filled accordingly. The capsules may be optionally banded for additional stability.

TABLE 3
CAPSULE ORAL COMPOSITIONS OF THE INVENTION

| | | | Spiro-oxindole | | Unit Dosage | | |
|---|---|---|---|---|---|---|---|
| | | | compound | | Amount | | |
| | | | Saturated | Maximum per | (mg/capsule) | | |
| | Excipient | mg Excipient/ | Solubility | capsule (mg/ | High | Mid | Low |
| Excipients | ratio | capsule | (mg/mL) | 500 μL) | Dose | Dose | Dose |
| Labrosol ® | 50 | 250 | 227 | 113 | 100 | 25 | 5 |
| Gelucire ® 44/14 | 40 | 200 | | | | | |
| Propylene glycol | 10 | 50 | | | | | |

In another embodiment of the invention, a process for the preparation of a capsule containing a pharmaceutical composition of the invention for oral administration to a mammal, wherein the pharmaceutical composition comprises a therapeutically effective amount of a spiro-oxindole compound of the invention, as a racemate, a single enantiomer, or a non-racemic mixture of enantiomers, or a pharmaceutically acceptable salt thereof, and one or more pharmaceutically effective excipients, is performed by first warming Imwitor® 742 in the amount listed in Table 4 below at 35° C. until it liquefies. Cremophor® ELP, Labrasol® and Vitamin E in the amounts listed in Table 4 below are then added to the liquefied Imwitor® 742 until a solution is obtained. The desired therapeutically effective amount of the spiro-oxindole compound of the invention, as a racemate, a single enantiomer, or a non-racemic mixture of enantiomers, or a pharmaceutically acceptable salt thereof, preferably COMPOUND B, or a pharmaceutically acceptable salt thereof, is added to the solution. The resultant solution is mixed for a suitable period of time, preferably for a period of time of between about 30 minutes and about 1 hour. Upon completion (when the spiro-oxindole compound of the invention, as a racemate, a single enantiomer, or a non-racemic mixture of enantiomers, or a pharmaceutically acceptable salt thereof, is completely dissolved), the resultant solution is gradually cooled down to a suitable temperature, preferably to a temperature of between about 30° C. and about 40° C. and filtered through a 0.7 micron filter. The filtrate is transferred into capsule-filling equipment. Capsules capable of containing the requisite volume of the pharmaceutical composition so prepared in order to administer a therapeutically effective amount of a spiro-oxindole compound of the invention, as a racemate, a single enantiomer, or a non-racemic mixture of enantiomers, or a pharmaceutically acceptable salt thereof, preferably hard gelatin capsules, are then filled accordingly. The capsules may be optionally banded for additional stability (i.e., to prevent leaking).

TABLE 4
CAPSULE ORAL COMPOSITIONS OF THE INVENTION

| | | Unit Dosage Amount (mg/capsule) | | |
|---|---|---|---|---|
| | | High Dose | Mid Dose | Low Dose |
| Components | Level (% w/w) | (100 mg) | (20 mg) | (10 mg) |
| lmwitor ® 742 | 9.0 | 75.0 | 15.0 | 7.5 |
| Cremophor ® ELP | 18.8 | 156.7 | 31.4 | 15.7 |
| Labrasol ® | 58.1 | 484.2 | 97.0 | 48.4 |
| Vitamin E, Liquid | 2.1 | 17.5 | 3.5 | 1.7 |
| COMPOUND B | 12.0 | 100.0 | 20.0 | 10.0 |
| Total | 100.0 | 833.3 | 167.0 | 83.3 |

The dose strengths are weight multiples of the same basic composition.

The processes described above can be carried out utilizing conventional equipment and under conventional conditions known to those skilled in the art.

Alternatively, a desired therapeutically effective amount of a spiro-oxindole compound of the invention, as a racemate, a single enantiomer, or a non-racemic mixture of enantiomers, or a pharmaceutically acceptable salt thereof, may be first mixed with an appropriate amount of cyclodextrin or a cyclodextrin-containing agent by methods known to one skilled in the art in order to further facilitate the solubility of the spiro-oxindole compound of the invention, as a racemate, a single enantiomer, or a non-racemic mixture of enantiomers, or a pharmaceutically acceptable salt thereof, when dissolved in the desired pharmaceutically acceptable excipients. The amount of cyclodextrin used is dependent upon the particular situation and can vary. While not intended to limit the scope of the invention in any way, the final concentration of the cyclodextrin in the pharmaceutical compositions of the invention can be from about 0.1% w/w to about 40% w/w.

In vivo Pharmacokinetic Profiles of the Pharmaceutical Compositions of the Invention

The in vivo pharmacokinetic profiles of the pharmaceutical compositions of the invention were determined as follows. Pharmaceutical compositions of the invention were orally administered to dogs in a controlled experiment to determine pharmacokinetic profile of a spiro-oxindole compound of the invention in a pharmaceutical composition of the invention.

Non-naïve male beagle dogs (Marshall Farms USA, Inc.) ranging in body weights from 6-10 Kg were used for the study. Each dog was fasted overnight before dosing. The fasted dogs (n=3/group) were given a single dose of 100 mg capsule (as setout in Table 4 above) or 400 mg (4×100 mg capsule) by oral administration (PO). Food was returned 4-hours post-dose. Blood samples were collected via jugular venipuncture at various timepoints (0.25, 0.1, 1, 2, 4, 6, 8, 24 and 48 hrs) after administration and plasma concentration was determined by liquid chromatography mass spectroscopy (LC-MS/MS). Concentrations of the active ingredient in the plasma samples at each timepoint were determined using standard methods known to one skilled in the art. The active ingredient concentrations were plotted against time (time in hours versus concentration in ng/mL) and the area under the curve extrapolated to infinity (AUC_{inf}), the Cₘₐₓ (peak plasma concentration of the active ingredient) and Tₘₐₓ (time after administration of the pharmaceutical composition when peak plasma concentration level occurs) were calculated.

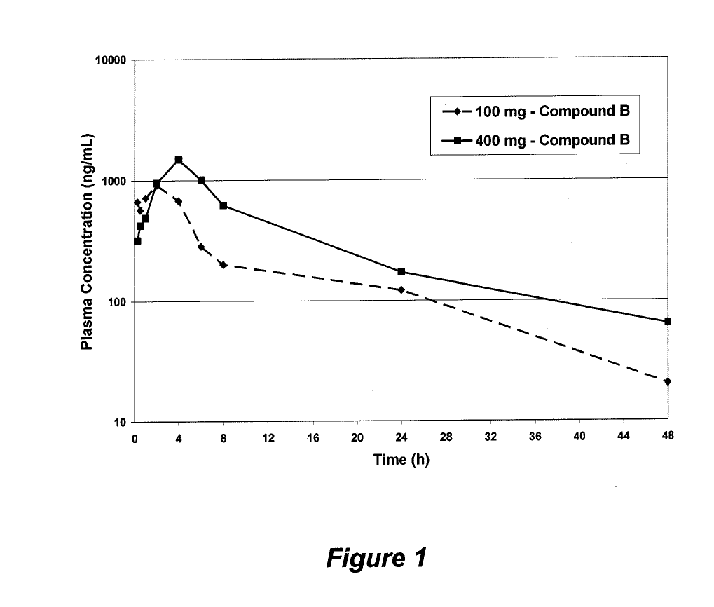

FIG. 1 shows the COMPOUND B plasma concentration-time profile for a single dose of the 100 mg or 400 mg given by PO administration. Following PO administration, COMPOUND B was readily absorbed with a Tₘₐₓ of 1 hour to 3 hours with suitable exposure levels for both 100 mg and 400 mg based formulations and hence a suitable therapeutic level. Also, the results indicate that COMPOUND B in the pharmaceutical formulation of the invention can achieve a suitable level of peak concentration (Cₘₐₓ) for both 100 mg and 400 mg based formulations (Cₘₐₓ was found to be in the range of 1300 to 1600 ng/mL for 100 mg and 400 mg).

### Administration of the Pharmaceutical Compositions of the Invention

The pharmaceutical compositions of the invention are to be orally administered to a mammal, preferably a human. The pharmaceutical compositions of the invention are formulated so as to allow the spiro-oxindole compound of the invention, as a racemate, a single enantiomer, or a non-racemic mixture of enantiomers, or a pharmaceutically acceptable salt thereof, contained therein to be bioavailable upon oral administration of the composition to the mammal. Pharmaceutical compositions of the invention that will be orally administered to a mammal take the form of one or more dosage units, where for example, a tablet or a capsule is considered a single dosage unit. Actual methods of preparing such dosage units are known, or will be apparent, to those skilled in this art; for example, see The Science and Practice of Pharmacy, 20th Edition (Philadelphia College of Pharmacy and Science, 2000). The pharmaceutical composition of the invention to be administered will, in any event, contain a therapeutically effective amount of a spiro-oxindole compound of the invention, as a racemate, a single enantiomer, or a non-racemic mixture of enantiomers, or a pharmaceutically acceptable salt thereof, for treatment of a disease or condition of interest in accordance with the teachings of this invention.

Typically, a successful therapeutic effective amount of an pharmaceutical composition of the invention for oral administration to a mammal, in need thereof, will meet some or all of the following criteria. Animal model efficacy of a spiro-oxindole compound of the invention, as a racemate, a single enantiomer, or a non-racemic mixture of enantiomers, or a pharmaceutically acceptable salt thereof, should be less than about 0.1 μg/Kg to about 100 mg/Kg body weight and the target human dose of a spiro-oxindole compound of the invention, as a racemate, a single enantiomer, or a non-racemic mixture of enantiomers, or a pharmaceutically acceptable salt thereof, should be between 0.1 μg/Kg to about 100 mg/Kg body weight, although doses outside of this range may be acceptable (“mg/Kg” means milligrams of compound per kilogram of body mass of the subject to whom it is being administered). The therapeutic index (or ratio of toxic dose to therapeutic dose) of a spiro-oxindole compound of the invention, as a racemate, a single enantiomer, or a non-racemic mixture of enantiomers, or a pharmaceutically acceptable salt thereof, should be greater than 100. The potency (as expressed by IC₅₀ value) of a spiro-oxindole compound of the invention, as a racemate, a single enantiomer, or a non-racemic mixture of enantiomers, or a pharmaceutically acceptable salt thereof, should be less than 10 μM, preferably below 1 μM and most preferably below 50 nM. The IC₅₀ (“Inhibitory Concentration—50%”) is the measure of the amount of a spiro-oxindole compound of the invention, as a racemate, a single enantiomer, or a non-racemic mixture of enantiomers, or a pharmaceutically acceptable salt thereof, required to achieve 50% inhibition of ion flux through a sodium channel, over a specific time period, in an assay designed to measure such flux. For example, COMPOUND A, or a pharmaceutically acceptable salt thereof, when tested in the guanidine influx assay disclosed in PCT Published Patent Application No. WO 06/110917 (see BIOLOGICAL EXAMPLE 1 therein), demonstrated an IC₅₀ of less than 1 μM concentration.

Therapeutically effective unit dosage amounts of a spiro-oxindole compound of the invention, as a racemate, a single enantiomer, or a non-racemic mixture of enantiomers, or a pharmaceutically acceptable salt thereof, in a pharmaceutical composition of the invention for oral administration to a mammal, preferably a human, are between about 0.1 mg and about 200 mg, between about 1.0 mg and about 150 mg, between about 5.0 mg and about 100 mg, and between about 20 mg and 50 mg. Preferably, a spiro-oxindole compound of the invention, as a racemate, a single enantiomer, or a non-racemic mixture of enantiomers, or a pharmaceutically acceptable salt thereof, is present in a pharmaceutical composition of the invention in a unit dosage amount of 5 mg, 10 mg, 15 mg, 25 mg or 100 mg. The ranges of therapeutically effective unit dosage amounts are not intended to be limiting. However, the most preferred unit dosage amount will be tailored to the individual mammal, as is understood and determinable by one skilled in the relevant arts (see, e.g., Berkowet al., eds., The Merck Manual, 16^{th} edition, Merck and Co., Rahway, N.J., 1992; Goodmanetna., eds., Goodman and Cilman's The Pharmacological Basis of Therapeutics, 10^{th} edition, Pergamon Press, Inc., Elmsford, N.Y., (2001); Avery's Drug Treatment: Principles and Practice of Clinical Pharmacology and Therapeutics, 3rd edition, ADIS Press, LTD., Williams and Wilkins, Baltimore, Md. (1987), Ebadi, Pharmacology, Little, Brown and Co., Boston, (1985); Osolci al., eds., Remington's Pharmaceutical Sciences, 18^{th} edition, Mack Publishing Co., Easton, Pa. (1990); Katzung, Basic and Clinical Pharmacology, Appleton and Lange, Norwalk, Conn. (1992)).

Alternatively, a spiro-oxindole compound of the invention, as a racemate, a single enantiomer, or a non-racemic mixture of enantiomers, or a pharmaceutically acceptable salt thereof, is present in an pharmaceutical composition of the invention for oral administration to a mammal, preferably a human, in a concentration of from about 0.1% w/w to about 25% w/w, preferably from about 0.5% w/w to about 20% w/w, more preferably from about 0.9% w/w to about 17% w/w.

The total dose required for each treatment can be administered by multiple doses or in a single dose over the course of the day, if desired. Generally, treatment is initiated with smaller dosages, which are less than the optimum dose of the active ingredient. Thereafter, the dosage is increased by small increments until the optimum effect under the circumstances is reached. The pharmaceutical composition can be orally administered alone or in conjunction with other pharmaceutically active agents directed to the treatment of the disease or condition or symptoms of the disease or condition.

### Biological Assays

Various techniques are known in the art to determine the safety and efficacy of the pharmaceutical compositions of the invention. In order that the invention described herein may be more fully understood, the following biological assays are set forth. It should be understood that these examples are for illustrative purposes only and are not to be construed as limiting this invention in any manner.

### BIOLOGICAL EXAMPLE 1

### Clinical Trial for Treatment of Pain from Primary/Inherited Erythromelalgia (IEM)

Primary/Inherited Erythromelalgia (IEM) is a rare inherited pain condition. The underlying cause of IEM can be one or more gain-of-function mutation(s) in the Naᵥ1.7 voltage-gated sodium channel, which COMPOUND B has been shown to inhibit.

Human patients with IEM have recurrent episodes of intense burning pain associated with redness and warmth in the hands and feet, but eventually the pain becomes constant. The pain is relieved by cooling, but has been largely resistant to pharmacological intervention. However, there are reports of voltage-gated sodium channel blockers showing moderate to outstanding pain relief for this condition.

A clinical trial for determining the efficacy of a pharmaceutical composition of the invention comprising COMPOUND B in ameliorating or alleviating IEM can be designed to be a three-period, double-blind, multiple-dose, and crossover study to minimize the dropout rate of participants, and will take into consideration that the patients enrolled will only be available for a 10-day study. Each patient enrolled in the study will serve as their own control, receiving both placebo and 400 mg of a pharmaceutical composition of the invention comprising COMPOUND B twice daily in a cross-over fashion.

### BIOLOGICAL EXAMPLE 2

### Clinical Trial for Treatment of Dental Pain

The purpose of this clinical trial was to compare the safety and efficacy (onset, duration of relief, and overall efficacy) of a single 500 mg dose of a pharmaceutical composition of the invention comprising COMPOUND B versus a placebo dose for relief of pain following extraction of impacted third molar teeth.

Sixty-one subjects were enrolled in the study. The mean age for the subjects was 20.4 years, and all subjects were male. The majority of subjects were Caucasians (95.1%).

The severity and relief of the pain was measured using an 11-point Pain Intensity Numerical Rating Scale (graded from 0=no pain at all to 10=worst pain imaginable) (PINRS) and a 5-point Categorical Pain Relief Scale (REL). Subjects completed the PINRS after surgery, but before the administration of (S)-enantiomer of the invention. Efficacy variables were derived from the REL and PINRS scores and included total pain relief (TOTPAR), pain intensity difference (PID), and summed pain intensity difference (SPID) and evaluated at time points of 4, 6, 8, and 12 hours after administration of the pharmaceutical composition of the invention comprising COMPOUND B.

The primary and all secondary endpoints showed a consistent analgesic trend with distinct separation of the pharmaceutical composition of the invention comprising COMPOUND B from placebo. These results suggest that the pharmaceutical composition of the invention comprising COMPOUND B has analgesic properties, but statistical significance from the placebo was not achieved due to two main reasons: (1) relatively high placebo response rate and (2) the slow onset of action of the pharmaceutical composition of the invention comprising COMPOUND B. The dental model utilized is designed and best suited for the evaluation of drugs with rapid onset such as the NSAID class of antiinflammatory agents. It was evident from this study that the pharmaceutical composition of the invention comprising COMPOUND B did not have such a NSAID-like rapid onset of action. However, the pain relief demonstrated by those subjects who received the pharmaceutical composition of the invention comprising COMPOUND B was higher compared to those subjects who only received the placebo, sufficiently so that the total efficacy population showed a consistent analgesic signal for all endpoints evaluated.

### BIOLOGICAL EXAMPLE 3

### Clinical Trial for Treatment of Post-Herpetic Neuralgia

Post Herpetic Neuralgia (PHN) is a well established and well recognized model for studying neuropathic pain. Furthermore, PHN demonstrates strong evidence of sodium channel blocker efficacy. The following study represents a randomized, double-blind, placebo-controlled, two-treatment, two-period cross-over study to evaluate the safety, tolerability, preliminary efficacy and systemic exposure of a pharmaceutical composition of the invention comprising COMPOUND B when orally administered to patients with PHN. The primary objectives are (a) to compare the safety and efficacy of a pharmaceutical composition of the invention comprising COMPOUND B to that of placebo for the relief of pain in patients with PHN, and (b) to evaluate the extent of systemic exposure of COMPOUND B following oral administration of a pharmaceutical composition of the invention comprising COMPOUND B in patients with PHN. The treatments will consist of a pharmaceutical composition of the invention comprising COMPOUND B and the matching placebo pharmaceutical composition.

The study may include the following four periods:

1. An initial screening and washout period (up to 3 weeks);

2. A single-blind, placebo run-in period (1 week);

3. A cross-over treatment period that will consist of 2 treatment periods each lasting 3 weeks separated by 2 weeks of washout/single-blind placebo run-in (total of 8 weeks); and

4. A safety follow-up period (2 weeks).

All of the U.S. patents, U.S. patent application publications, U.S. patent applications, foreign patents, foreign patent applications and non-patent publications referred to in this specification and/or listed in the Application Data Sheet are incorporated herein by reference, in their entirety.

Although the foregoing invention has been described in some detail to facilitate understanding, it will be apparent that certain changes and modifications may be practiced within the scope of the appended claims. Accordingly, the described embodiments are to be considered as illustrative and not restrictive, and the invention is not to be limited to the details given herein, but may be modified within the scope and equivalents of the appended claims.

1. A pharmaceutical composition for oral administration to a mammal comprising one or more pharmaceutically acceptable excipients and a therapeutically effective amount of a spiro-oxindole compound having the following formula (I): 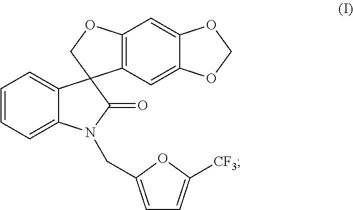
as a racemate, a single enantiomer, or a non-racemic mixture of enantiomers, or a pharmaceutically acceptable salt thereof.

2. The pharmaceutical composition of claim 1 comprising two or more pharmaceutically acceptable excipients.

3. The pharmaceutical composition of claim 2 wherein the pharmaceutically acceptable excipients are selected from the group consisting of Miglyol® 840, Labrafac®, Captex® 200P, Myvacet® 9-45K, PEG 400, Capmul® PG8, TPGS, Neobee® M-5, Transcutol®, Capryol® 90, Solutol® HS 15, Corn Oil Labrasol®, Capryol® 90, Gelucire® 44/14, a cyclodextrin, PEG 400, PEG 6000, ethanol, water, propylene glycol, Cremophor ELP®, Imwitor® 742, Vitamin E and Polyvinylpyrrolidone (PVP).

4. The pharmaceutical composition of claim 3 wherein the pharmaceutically acceptable excipients are selected from the group consisting of Labrasol®, Gelucire® 44/14 and propylene glycol.

5. The pharmaceutical composition of claim 3 wherein the pharmaceutically acceptable excipients are selected from the group consisting of Labrasol®, Cremophor® ELP, Imwitor® 742, Vitamin E and PVP.

6. The pharmaceutical composition of claim 4 wherein each pharmaceutically acceptable excipient is present in a concentration of from about 0.1% w/w to about 99% w/w.

7. The pharmaceutical composition of claim 6 wherein Labrasol® is present in a concentration of from about 30% to about 70% w/w, Gelucire® 44/14 is present in a concentration of from about 20% to about 50% w/w and propylene glycol is present in a concentration of from about 0.5% to about 20% w/w.

8. The pharmaceutical composition of claim 7 wherein Labrasol® is present in a concentration of from about 35% to about 65% w/w, Gelucire® 44/14 is present in a concentration of from about 25% to about 45% w/w and propylene glycol is present in a concentration of from about 1.0% to about 10% w/w.

9. The pharmaceutical composition of claim 5 wherein each pharmaceutically acceptable excipient is present in a concentration of from about 0.1% w/w to about 99% w/w.

10. The pharmaceutical composition of claim 9 wherein Labrasol® is present in a concentration of from about 30% to about 70% w/w, Cremophor ELP® is present in a concentration of from about 20% to about 50% w/w, Imwitor® 742 is present in a concentration of from about 0.5% to about 10% w/w, Vitamin E is present in a concentration of from about 0.1% to about 5% w/w and PVP is present in a concentration of from about 0.5% to about 10% w/w.

11. The pharmaceutical composition of claim 1 wherein the spiro-oxoindole compound is present in a concentration of from about 0.1% w/w to about 25% w/w.

12. The pharmaceutical composition of claim 1 in a capsule form containing the spiro-oxindole compound in a unit dosage amount of between about 5 mg to about 100 mg.

13. The pharmaceutical composition of claim 1 wherein the pharmaceutical composition is in liquid form.

14. A pharmaceutical composition for oral administration to a mammal comprising one or more pharmaceutically acceptable excipients and a therapeutically effective amount of a spiro-oxindole compound having the following formula (I): 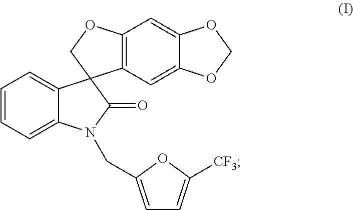
as a racemate, a single enantiomer, or a non-racemic mixture of enantiomers, or a pharmaceutically acceptable salt thereof;
wherein the spiro-oxindole compound is present in a concentration of from about 0.1% w/w to about 25% w/w, wherein a first pharmaceutically acceptable excipient is Labrasol® and is present in a concentration of from about 35% w/w to about 65% w/w, wherein a second pharmaceutically acceptable excipient is Gelucire® 44/14 and is present in a concentration of from about 25% w/w to about 45% w/w, and wherein a third pharmaceutically acceptable excipient is propylene glycol and is present in a concentration of from about 1.0% w/w to about 10% w/w.

15. A pharmaceutical composition for oral administration to a mammal comprising one or more pharmaceutically acceptable excipients and a therapeutically effective amount of a spiro-oxindole compound having the following formula (I): 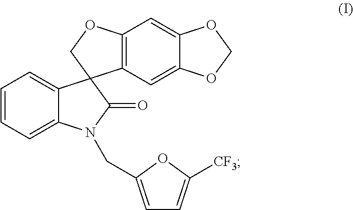
as a racemate, a single enantiomer, or a non-racemic mixture of enantiomers, or a pharmaceutically acceptable salt thereof;
wherein the spiro-oxindole compound is present in an unit dosage amount of between about 5 mg and about 100 mg, wherein a first pharmaceutically acceptable excipient is Labrasol® and is present in a concentration of from about 35% w/w to about 65% w/w, wherein a second pharmaceutically acceptable excipient is Gelucire® 44/14 and is present in a concentration of from about 25% w/w to about 45% w/w, and wherein a third pharmaceutically acceptable excipient is propylene glycol and is present in a concentration of from about 1.0% w/w to about 10% w/w.

16. The pharmaceutical composition of claim 1 wherein the spiro-oxindole compound is the (S)-enantiomer of the compound of formula (I) having the following formula (I-S):

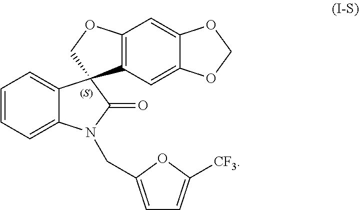

17. A method of treating a sodium channel-mediated disease or condition in a mammal, wherein the method comprises orally administering to the mammal in need thereof a therapeutically effective amount of a pharmaceutical composition comprising one or more pharmaceutically acceptable excipients and a therapeutically effective amount of a spiro-oxindole compound having the following formula (I): 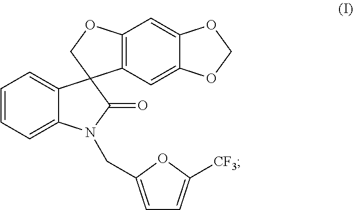
as a racemate, a single enantiomer, or a non-racemic mixture of enantiomers, or a pharmaceutically acceptable salt thereof.

18. The method of claim 17, wherein said disease or condition is selected from the group consisting of pain associated with HIV, HIV treatment induced neuropathy, trigeminal neuralgia, post-herpetic neuralgia, diabetic neuropathy, peripheral neuropathy, Complex regional pain syndrome, Paroxysmal Extreme Pain Disorder, eudynia, familial erythromelalgia, secondary erythromelalgia, primary/inherited erythromelalgia, familial rectal pain, familial facial pain, dental pain, migraine, headache, familial hemiplegic migraine, sinus headache, tension headache, phantom limb pain, peripheral nerve injury, pain associated with multiple sclerosis (MS); myasthenia syndromes, myotonia, paroxysmal dystonia, periodic paralysis, spasticity, spastic paraplegia, myopathies, paramyotonia congentia, hyperkalemic periodic paralysis, hypokalemic periodic paralysis, malignant hyperthermia, heat sensitivity, irritable bowel syndrome, Crohns disease, motor impairment associated with MS, amyotrophic lateral sclerosis (ALS), pruritis, benign prostatic hyperplasia, arthritis, rheumatoid arthritis, osteoarthritis, cystic fibrosis, pseudoaldosteronism, rhabdomyolysis, bipolar depression, anxiety, schizophrenia, illness due to exposure to insecticides or other sodium channel toxins, cancer, epilepsy, partial and general tonic seizures, restless leg syndrome, fibromyalgia, neuroprotection under ischaemic conditions caused by stroke, glaucoma or neural trauma, arrhythmias, long-QT syndrome, Catecholeminergic polymorphic ventricular tachycardia, tachy-arrhythmias, atrial fibrillation and ventricular fibrillation.

19. The method of claim 18, wherein said disease or condition is primary/inherited erythromelalgia.

20. The method of claim 18 wherein the disease or condition is post-herpetic neuralgia.

21. A method of treating pain through inhibition of ion flux through a voltage-dependent sodium channel in a mammal, wherein the method comprises orally administering to the mammal in need thereof a therapeutically effective amount of a pharmaceutical composition comprising one or more pharmaceutically acceptable excipients and a therapeutically effective amount of a spiro-oxindole compound having the following formula (I): 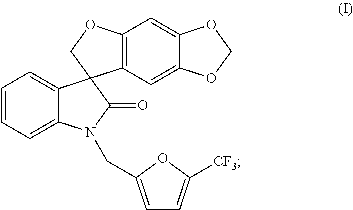
as a racemate, a single enantiomer, or a non-racemic mixture of enantiomers, or a pharmaceutically acceptable salt thereof.

22. A method of treating benign prostatic hyperplasia in a mammal, wherein the method comprises orally administering to the mammal in need thereof a therapeutically effective amount of a pharmaceutical composition comprising one or more pharmaceutically acceptable excipients and a therapeutically effective amount of a spiro-oxindole compound having the following formula (I): 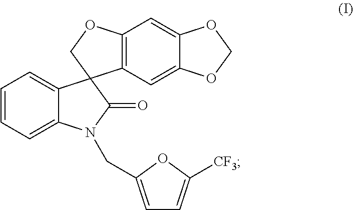
as a racemate, a single enantiomer, or a non-racemic mixture of enantiomers, or a pharmaceutically acceptable salt thereof.

23. A method of treating pruritis in a mammal, wherein the method comprises orally administering to the mammal in need thereof a therapeutically effective amount of a pharmaceutical composition comprising one or more pharmaceutically acceptable excipients and a therapeutically effective amount of a spiro-oxindole compound having the following formula (I): 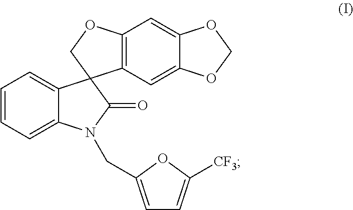
as a racemate, a single enantiomer, or a non-racemic mixture of enantiomers, or a pharmaceutically acceptable salt thereof.

24. A method of treating cancer in a mammal, wherein the methods comprises orally administering to the mammal in need thereof a therapeutically effective amount of a pharmaceutical composition comprising one or more pharmaceutically acceptable excipients and a therapeutically effective amount of a spiro-oxindole compound having the following formula (I): 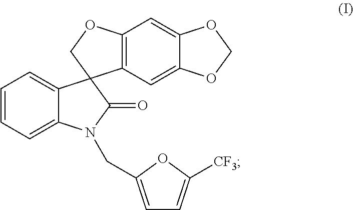
as a racemate, a single enantiomer, or a non-racemic mixture of enantiomers, or a pharmaceutically acceptable salt thereof.

25. The method of claim 17 wherein the spiro-oxindole compound is the (S)-enantiomer of the compound of formula (I) having the following formula (I-S): 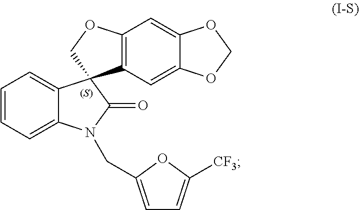
or a pharmaceutically acceptable salt thereof.
